SEQUENCE LISTING (8 sequences)

SEQ ID NO: 1 - DNA, 1099 bp, artificial sequence
  the DNA sequence of the PD-L1 nanobody
cggggcgggaacatttccaagcttaaggagacagtacatatgaaatacctattgcctacggcagccgctggattgttattactcgcggcccagccggccatggcccaggtgcagctgcaggagtctgggggaggcttggtgcaggctgggggctctctgagactctcctgtgcagcctctggacgcaccttcagaaacgatgtcatggcctggttccgccagattccagggaaggagcgtgagtttgttgcggtgattgcctacgatgcggctgacacagactacgcagactccgtgaagggccgattcatcatctccagagacaacgccaagaacacgatatatttgcaaatgaacaccctgaaacctgaggacacggccgtttattactgtgcagccgacaaggacagaatgtacggtagtaggcactggccggaatatgagtatgactactggggccaggggacccaggtcaccgtctcctcagcggccgcatacccgtacgacgttccggactacggttcccaccaccatcaccatcactagactgttgaaagttgtttagcaaaacctcatacagaaaattcatttactaacgtctggaaagacgacaaaactttagatcgttacgctaactatgagggctgtctgtggaatgctacaggcgttgtcgtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacagcacttatccgcctggtactggagcaaaaccccgctaatcctaaatccttctcttggaggagtctcagcctcttaatactttcatgtttcagaataataggtccgaataaggcagggtgcataagctgtttatacgggactgttactcaaggcactgacccgattaaagttagtaacagtacactcccgtgaatcatacgaagcatggtaggacgcttaactgggacaggaaaagtc

SEQ ID NO: 2 - DNA, 17 bp, artificial sequence
  PD-L1 upstream primer
gacacgaattcgccacc

SEQ ID NO: 3 - DNA, 24 bp, artificial sequence
  PD-L1 downstream primer
gtgtcaagctttcacttatcatca

SEQ ID NO: 4 - DNA, 23 bp, artificial sequence
  Primer CAL-leader sequence
gtcctggctgctcttctacaagg

SEQ ID NO: 5 - DNA, 23 bp, artificial sequence
  Primer CA-CH2 sequence
ggtacgtgctgttgaactgttcc

SEQ ID NO: 6 - DNA, 29 bp, artificial sequence
  Primer VHH-back sequence
gatgtgcagctgcaggagtctggrggagg

SEQ ID NO: 7 - DNA, 34 bp, artificial sequence
  Primer PMCF sequence
ctagtgcggccgctgaggagacggtgacctgggt

SEQ ID NO: 8 - DNA, 20 bp, artificial sequence
  primer MP57
ttatgcttccggctcgtatg

(19) United States
(12) Patent Application Publication
(10) Pub. No.: US 2021/0002370 A1
(43) Pub. Date: Jan. 7, 2021

(54) PD-L1 NANOBODIES, PREPARATION METHODS AND USES THEREOF

(71) Applicant: Shihezi University, Shihezi City (CN)

(72) Inventors: Chuangfu CHEN, Shihezi City (CN); Peng WU, Shihezi City (CN)

(21) Appl. No.: 16/920,551

(22) Filed: Jul. 3, 2020

(30) Foreign Application Priority Data
Jul. 5, 2019 (CN) 201910604225.0
Mar. 9, 2020 (CN) 202010156943.9

(51) Int. Cl. C07K 16/28; C12N 15/10; C12Q 1/686

(52) U.S. Cl. CPC C07K 16/2827; C12Q 1/686; C12N 15/1037

(57) ABSTRACT

This disclosure pertains to the technical field of antibodies, and discloses PD-L1 nanobodies, preparation methods and uses thereof; two primers are designed and synthesized; a sufficient amount of target products is amplified by PCR to carry out replacement enzyme digestion; target fragments are ligated to vectors, transformed, and screened to obtain clones; proteins are identified and expressed; antibody library is constructed; M13 phage display system is selected to display VHH antibody library, which consists of pMECS phagemid vector, E. coli TG1 and M13K07 helper phage. In the phagemid vector pMECS of this disclosure, the sequence before the Pst I restriction site is the coding sequence of the pelB secretion signal peptide and part of the amino acids in the first framework region of the antibody; the pelB signal peptide can guide the secretion of the subsequent polypeptide to the periplasmic cavity; Not I restriction site is followed by the coding sequence of HA and 6×His tag, which can be used for purification or detection of fusion protein.

### TECHNICAL FIELD

The disclosure pertains to the technical field of antibodies, and particularly relates to PD-L1 nanobodies, preparation methods and uses thereof.

### BACKGROUND

At present, some monoclonal antibodies against PD-L1 have been approved for cancer treatment. For example, PD-L1 inhibitor Tecentriq (called Atezolizumab or MDL3280a) is approved for the treatment of bladder cancer (urothelial carcinoma). PD-L1 has an increased expression in most tumor tissues, such as breast cancer, non-small cell lung cancer and melanoma. The expression of PD-L1 in tumor cells is also considered to be a prognostic factor for many types of malignant tumors. In order to block the binding of PD-L1 to the receptor PD1 and inhibit the development of tumor, it is very fast to develop immunotherapeutic antibody drugs, and well-known drugs such as opdivo, keytruda and Atezolizumab also have significant effects. Therefore, before using these drugs, it is necessary to perform PD-L1 gene decoding and gene testing. Professor Zhou Caicun from Shanghai Pulmonary Hospital, also director of Oncology Research Institute of Tongji University School of Medicine, pointed out that current clinical trial evidence shows that PD-1 immune checkpoint inhibitors, such as Pembrolizumab, all show better efficacy when used as first-line single drugs or in combination with chemotherapy for the treatment of non-small cell lung cancer without EGFR/ALK gene mutations than standard chemotherapy alone, and bring more significant survival benefits to patients. However, when judging whether it is monotherapy or combination chemotherapy, we need to make a decision based on the expression of tumor biomarker PD-L1 in order to achieve more accurate results. At present, there are four PD-L1 detection kits corresponding to immunotherapy in clinical research abroad: Roche's SP142 (for PD-L1 detection of Roche's Atezolizumab), Roche's SP263 (for PD-L1 detection of AstraZeneca's Durvalumab), Dako's 28-8 (for PD-L1 detection of Squibb's Nivolumab), Dako's 22C3 (for PD-L1 detection of MSD's Pembrolizumab).

Compared with traditional chemotherapy or targeted therapy, biological immunotherapy has an essential logical difference: “immunotherapy” targets immune cells (or immune system) rather than cancer cells. In cancer immunotherapy, suppression of immune checkpoint pathways is considered to be one of the most promising treatments. Its mechanism is to release the suppressed state of T cell activity by inhibiting relevant targets in pathways, and activated T cells can attack and eliminate tumor cells. Antibodies do not directly act on tumor cells, but indirectly kill tumor cells by acting on T cells; in addition, they are not against a specific substance on the surface of tumor, but systematically enhance the anti-tumor immune response throughout the body.

Nanobodies were first reported by a Belgian scientist in the journal Nature in 1993. There is an antibody that naturally lacks light chains in the peripheral blood of alpaca. The antibody contains only one heavy chain variable region (VHH) and two conventional CH2 and CH3 regions, but are not as easily adhered to each other as artificially modified single chain antibody fragments (scFv), or even aggregated into blocks. More importantly, the VHH structure cloned and expressed separately has structural stability and antigen-binding activity comparable to those of the original heavy chain antibody, and is currently known as the smallest unit that can bind to a target antigen. VHH crystal is 2.5 nm, 4 nm long, and its molecular weight is only 15 KD, so it is also called Nanobody (Nb).

In view of the above, problems in the prior art include:

(1) Monoclonal antibodies are heterologous. If a monoclonal antibody which is used as a mouse monoclonal antibody is applied to a human body, it will produce an anti-mouse monoclonal antibody, and it cannot be repeatedly applied, which affects its efficacy.

(2) Monoclonal antibodies are relatively large in size and cannot enter tumor tissues effectively.

(3) Monoclonal antibodies have a long development cycle, high production costs, and low output.

(4) Monoclonal antibody drugs are expensive, complex in research and development, complex in humanization, and limited in success rate.

(5) Monoclonal antibody drugs are difficult to produce on a large scale, and require large investments in factory construction and production.

(6) Monoclonal antibody drugs are unstable, easy to decompose, and high in preservation cost; easy to pollute, and high in maintenance cost. The drugs will decompose under conditions of high temperature and strong acid and alkali, and must be stored at low temperature; otherwise, they will completely lose their activity within a few weeks. Antibodies can be quickly decomposed by digestive system, which prevents them from entering brain or other effective action sites.

The significance of solving the above technical problems:

A nanobody contains 3 hypervariable regions and 4 framework regions, and all of the hypervariable regions are on the same side. It has the same structure as the VH of a human antibody. Sequencing shows that it has extremely high homology with VH3, but the CDR1 (Complementarity-determining region-1) and CDR3 (Complementarity-determining region-3) of nanobodies are relatively long. The CDR3 of nanobodies has protrusion, which can increase the affinity of binding to an antigen. Nanobodies have a stable structure, which ensures the stability of binding. Phage display technology is a method commonly used to produce nanobodies. The sequence of nanobodies is introduced into the phage sequence. The target protein will be expressed on the phage shell. The construction of a phage library comprises immunizing a camelid, obtaining animal leukocytes, subjecting RNA to reverse transcription, and constructing a library specific for antigens.

Nanobodies can maintain their conformation in harsh environments. Nanobodies are particularly heat resistant and can be stored at room temperature for more than a week. The super acid and alkali resistance allows nanobodies to better resist different environments, and also increases the action range of nanobodies. The small size of a single domain heavy chain antibody also allows it to have low immunogenicity, making it possible that an animal can have a long-term injection of protein. The binding site of nanobodies to antigens is also different from that of monoclonal antibodies. Nanobodies can bind more tightly to antigens and can bind to places where traditional antigens cannot. The Chinese Academy of Medical Sciences has developed a “monoclonal antibody-drug” conjugate with targeted and selective killing of tumor cells, that is, monoclonal antibodies are used as “carriers” to carry drugs, accurately bind to tumor cells, and kill in situ cancer cells without damaging other normal cells. This comprehensive drug is like a “bio-missile” that specifically attacks cancer. This therapeutic method is figuratively called missile therapy.

### SUMMARY

In view of the problems in the prior art, this disclosure provides a method for preparing PD-L1 nanobodies from an alpaca immunized with PD-L1 antigens.

This disclosure is achieved by a nanobody, which is a PD-L1 nanobody having a sequence of SEQ ID NO: 1.

A method for preparing PD-L1 nanobodies from an alpaca immunized with PD-L1 antigens expressed in mammalian cells comprising:

Expressing PD-L1 antigens in transiently transfected mammalian cells, and screening to obtain nanobody fragment SEQ ID NO: 1 by biotinylation. Further, the method for preparing PD-L1 nanobodies from an alpaca immunized with PD-L1 antigens expressed in mammalian cells further comprises:

Step 1: construction of a vector: amplification of a target fragment, replacement enzyme digestion, ligation of target fragment and vector, transformation, and screening of clones.

Step 2: protein identification and expression.

Step 3: construction of an antibody library: total RNA extraction and cDNA synthesis of samples, preparation of VHH library fragments, electrotransformation and library construction.

Step 4: biotinylation screening and prokaryotic expression.

Further, in step 1, the amplification of the target fragment comprises:

(1) Designing Synthetic Primers:

PD-L1 upstream primer

| | |
|---|---|
| 5′-GACACGAATTCGCCACC-3′. | SEQ ID NO: 2 |

PD-L1 downstream primer

| | |
|---|---|
| 5′-GTGTCAAGCTTTCACTTATCATCA-3′. | SEQ ID NO: 3 |

Amplifying a sufficient amount of target product (PD-L1 Uniprot: Q9NZQ7) by PCR.

(2) Using a pfu High-Temperature Polymerase for PCR Reaction.

Further, in step 1, the amount of components of PCR is as follows:

The primer concentration is 1 OD soluble in 400 μl of ddH₂O.

| | |
|---|---|
| Reaction system | 50 μl |
| Primer mix | (1/34) 0.4 μl × 13.6 |
| | 8 μl in total |
| 10 × pfu Buffer | 5 μl |
| Upstream and downstream | 2 μl each |
| primers of each segment | |
| Pfu | 0.4 μl (5 U/μl) |
| ddH₂O | To 50 μl |

Further, in step 1, the specific steps of PCR amplification of target fragment include:

(1) A First Round PCR Procedure:

| | | |
|---|---|---|
| 95° C. 3 min | | |
| 95° C. 22 sec | | |
| 50° C. 20 sec | {close oversize brace} | 18 cycles. |
| 72° C. 40 sec | | |
| 72° C. 5 min | | |

The above is a first round PCR reaction system, and the first round PCR product is used as the template for a second round PCR.

(2) A Second Round PCR Reaction System:

The amount of components of PCR is as follows: The primer concentration is 1 OD soluble in 400 μl of ddH₂O.

| | |
|---|---|
| Upstream primer-1 | 2 μl |
| Downstream primer-34 | 2 μl |
| First round PCR product | 1 μl |
| dNTP | 1 μl (25 mM each) |
| 10 × pfu Buffer | 5 μl |
| Pfu | 0.4 μl (5 U/μl) |
| ddH₂O | To 50 μl |

(3) A Second Round PCR Procedure:

| | | |
|---|---|---|
| 95° C. 3 min | | |
| 95° C. 22 sec | | |
| 55° C. 20 sec | {close oversize brace} | 22 cycles. |
| 72° C. 45 sec | | |
| 72° C. 5 min | | |

The second round of PCR is subjected to agarose gel electrophoresis to recover purified fragments for enzyme digestion.

Further, in step 1, replacement enzyme digestion is carried out, and the above PCR product is digested.

| | |
|---|---|
| PCR product fragment digestion system | 50 μl |
| Recovered purified fragment | 1 μl (20 μl) |
| 10 × 10 × FD Buffer | 5 μl |
| EcoRI | 1 μl (10 U/μl) |
| HindIII | 1 μl (10 U/μl) |
| ddH₂O | 23 μl |

The above system is reacted in a 37° C. constant temperature water bath for 2 h.

Enzyme Digestion System of Vector:

| | | |
|---|---|---|
| PCDNA3.1+ | 1 | μg |
| 10 × FD Buffer | 5 | μl |
| EcoRI | 1 | μl (10 U/μl) |
| HindIII | 1 | μl (10 U/μl) |
| ddH2O | 42 | μl |

The above system is reacted in a 37° C. constant temperature water bath for 2 h.

The digested vectors and fragments are recovered.

Further, in step 1, the recovered purified target DNA fragments are ligated to vectors.

Ligation system: 20 μl.

Enzyme-digested target fragment: 8 μl.

Enzyme-digested vector PCDNA3.1+4 μl.

10×T4 DNAligase Buffer 2 μl.

T4 DNAligase 1 μl (5 U/μl).

ddH₂O supplemented to 20 μl.

The above ligation mix solution is placed in a PCR machine at 22° C. for 1 h.

Further, in step 1, transformation and screening of clones are carried out:

The above ligation solution is transferred into oneshort competent cells to detect and screen positive clones for sequencing.

Further, the DNA sequence of PD-L1 nanobody is:

SEQ ID NO: 1
CGGGGCGGGAACATTTCCAAGCTTAAGGAGACAGTACATATGAAATACCT
ATTGCCTACGGCAGCCGCTGGATTGTTATTACTCGCGGCCCAGCCGGCCA
TGGCCCAGGTGCAGCTGCAGGAGTCTGGGGGAGGCTTGGTGCAGGCTGGG
GGCTCTCTGAGACTCTCCTGTGCAGCCTCTGGACGCACCTTCAGAAACGA
TGTCATGGCCTGGTTCCGCCAGATTCCAGGGAAGGAGCGTGAGTTTGTTG
CGGTGATTGCCTACGATGCGGCTGACACAGACTACGCAGACTCCGTGAAG
GGCCGATTCATCATCTCCAGAGACAACGCCAAGAACACGATATATTTGCA
AATGAACACCCTGAAACCTGAGGACACGGCCGTTTATTACTGTGCAGCCG
ACAAGGACAGAATGTACGGTAGTAGGCACTGGCCGGAATATGAGTATGAC
TACTGGGGCCAGGGGACCCAGGTCACCGTCTCCTCAGCGGCCGCATACCC
GTACGACGTTCCGGACTACGGTTCCCACCACCATCACCATCACTAGACTG
TTGAAAGTTGTTTAGCAAAACCTCATACAGAAAATTCATTTACTAACGTC
TGGAAAGACGACAAAACTTTAGATCGTTACGCTAACTATGAGGGCTGTCT
GTGGAATGCTACAGGCGTTGTCGTTTGTACTGGTGACGAAACTCAGTGTT
ACGGTACATGGGTTCCTATTGGGCTTGCTATCCCTGAAAATGAGGGTGGT
GGCTCTGAGGGTGGCGGTTCTGAGGGTGGCGGTTCTGAGGGTGGCGGTAC
TAAACCTCCTGAGTACGGTGATACACCTATTCCGGGCTATACTTATATCA
ACCCTCTCGACAGCACTTATCCGCCTGGTACTGGAGCAAAACCCCGCTAA
TCCTAAATCCTTCTCTTGGAGGAGTCTCAGCCTCTTAATACTTTCATGTT
TCAGAATAATAGGTCCGAATAAGGCAGGGTGCATAAGCTGTTTATACGGG
ACTGTTACTCAAGGCACTGACCCGATTAAAGTTAGTAACAGTACACTCCC
GTGAATCATACGAAGCATGGTAGGACGCTTAACTGGGACAGGAAAAGTC.

Further, step 3 includes:

Four VHH library fragments are amplified using camel antibody primer pairs, respectively. The four VHH library fragments are inserted into pMECS phagemid vectors respectively and introduced into E. coli TG1 to construct a phage display antibody immune library with a storage capacity of >10⁹. Twenty clones are randomly picked from the library for sequence determination and analysis, so that more than 99% of the clones in the library comprise the target insert sequence.

Further, the preparation of VHH library fragments comprises: amplifying camel antibody fragments with primers CAL-leader and CAL-CH2 using cDNA as a template, respectively, and subjecting an appropriate amount of PCR product to 1% agarose gel electrophoresis detection.

Ligation and Transformation Pretest:

Before the formal establishment of the library, the quality of phagemid vectors, ligation efficiency of vectors and VHH library fragments are tested and evaluated by ligation and transformation pretest. VHH fragments digested with Pst I/Not I and phagemid vectors pMECS digested with the same Pst I/Not I are subjected to a ligation reaction at different ratios using T4 DNA ligases, and then transformed into E. coli TG1 chemically competent cells, which are coated on ampicillin-resistant plates for colony counting.

Electrotransformation and Library Construction:

According to the optimal ratio obtained by ligation and transformation pretests, vectors are ligated with four VHH fragments. The purified ligation products are electrotransformed into E. coli TG1 to obtain 15 ml of transformation product. 10 μl is taken and subjected to 10-fold gradient dilution, and three gradients, 10⁻⁴, 10⁻⁵ and 10⁻⁶, are used for counting on ampicillin-resistant plates to evaluate the library capacity, the library capacity=number of clones×dilution multiple×total volume of transformation products. The remaining transformation products are coated on 15 Amp-resistant plates with a diameter of 15 cm, cultured overnight, scraped off from each plate the next day, mixed evenly, to which 20% final concentration glycerol is added, packed in aliquots and cryopreserved at −80° C.

Quality Analysis of Immune Library:

Twenty monoclonals are randomly picked from the gradient dilution plate of each library, and colony PCR is performed using primers MP57 and PMCF. These clones are sequenced using primer MP57.

Further, step 4 includes:

1) PD-L1 Serum Titer Test:

The serum titer of PD-L1 before and after immunization is detected by gradient dilution ELISA method comprising coating with PD-L1, adding with gradient diluted antiserum, using a secondary antibody which is anti-alpaca-HRP 1:15 000 dilution, and finally developing with a TMB substrate.

2) PD-L1 Protein Sample SDS-PAGE, Western-Blot and Biotin Labeling:

The loading volume of PD-L1 protein for SDS-PAGE detection is 2 which is subjected to Western-blot detection, antiserum 1:20 000 dilution, and the anti-alpaca-HRP secondary antibody has a working concentration of 1:2 000, and developed by chemiluminescence.

3) Biotin Labeling and Efficiency Detection of Target PD-L1:

PD-L1 is labeled with biotin under the conditions of 0.25 mg/ml, pH 7.4, protein to biotin ratio of 1:15, and labeled at room temperature for 1 h. The labeled protein is subjected to a PD-Midi desalting column to remove free biotin, and the buffer is replaced with PBS 5% glycerol pH 7.4, and finally packed in aliquots and stored at −70° C.

Detection of biotin labeling efficiency of PD-L1: Two samples of 1.5 μg labeled b-PD-L1 protein are used, and then 5 μg streptavidin and 5 μl PBS are added, respectively. In addition, 5 μg of SA is also used, to which 5 μl of PBS is added, as a SA sample control. After reacting at room temperature for 1 h, 5 μl of non-reducing loading buffer is added to each sample, and directly subjected to SDS-PAGE without denaturation by heating.

4) In Vitro Targeted Screening:

PD-L1 is screened for 3 rounds using the constructed immune library.

5) Identification:

322 clones picked from the enriched products in the second and third rounds of elution are verified by Monoclonal phage ELISA and coated with PD-L1 and BSA control 200 ng/well, respectively.

16 unique clones of PD-L1 sequence are expressed by IPTG induction at 30° C. After centrifugation, the bacterial cells are collected and subjected to periplasmic cavity extraction. The periplasmic cavity extract sample is diluted 10-fold with 0.5×blocker and added to the coated and blocked PD-L1 and BSA; at the same time, the TG1 periplasmic cavity extract is set as a negative control. Mouse anti-HA tag 1:5 000 diluted monoclonal antibody is used as a secondary antibody, and 1:5 000 diluted goat anti-mouse-HRP is used as a tertiary antibody to detect the activity of a soluble expressed nanobody.

Another object of this disclosure is to provide a nanobody-drug conjugate constructed by combining with the PD-L1 nanobody prepared in the method.

A further object of this disclosure is to provide the use of the nanobody according to claim 1 in the preparation of a reagent for tumor detection or treatment.

A still further object of this disclosure is to provide the use of the nanobody according to claim 1 in the preparation of an immune adjuvant for improving the immune level of an animal or of an immune promoting agent during the transmission of viruses and/or bacteria.

In summary, the advantages and positive effects of this disclosure include:

In this disclosure, the PD-L1 antigen is transiently transfected and expressed in mammalian cells; the immunized animal is alpaca; screening is carried out by biotinylation; and the nanobody fragments obtained by screening have their own unique gene sequences. The antibody can be used to bind to a human target to block the binding of a signaling pathway, and can be used as a tumor therapy, tumor detection, etc.

The binding site of nanobodies and antigens is different from that of monoclonal antibodies. Using nanobodies instead of monoclonal antibodies can improve or synergistically enhance the binding to antigens. Nanobodies do not have a complete antibody structure, lacking the Fc end and Y-shaped structure, which makes nanobodies difficult to be recognized and can easily escape the capture of the immune system.

In this disclosure, human HEK293 cell strain is used to express antigens, and using a mammalian expression system to express a human protein can maximally maintain the original structure of the protein and ensure that the protein has modifications specific for eukaryotic proteins such as post-translational modifications, glycosylation and the like, allowing the obtained protein to have higher activity. This method maximally guarantees the original structure and activity of the protein.

The method of this disclosure uses alpaca as the immunized animal to better ensure and save the amount of antigens. Nanobodies obtained by screening according to this method can efficiently and specifically bind to the target site.

### BRIEF DESCRIPTION OF THE DRAWINGS

FIG. 1 is a flowchart of a method for preparing PD-L1 nanobodies from an alpaca immunized with PD-L1 antigens expressed in mammalian cells according to an example of this disclosure.

FIG. 2 is an original diagram of PCR agarose gel electrophoresis according to an example of this disclosure.

FIG. 3 is an agarose gel electrophoresis diagram of vector PCDNA3.1+ after enzyme digestion according to an example of this disclosure.

FIG. 4 is a schematic diagram of a sequence encoding phage PIII capsid protein according to an example of this disclosure.

FIG. 5 is a diagram of phagemid pMECS plasmids according to an example of this disclosure.

FIG. 6 is a diagram of PD-L1 PCR amplification according to an example of this disclosure.

FIG. 7 is a first diagram of identification result of enzyme digestion according to an example of this disclosure.

FIG. 8 is a second diagram of identification of PD-L1 enzyme digestion according to an example of this disclosure.

FIG. 9 is a diagram of detection result of PD-L1 SDS-PAGE according to an example of this disclosure.

FIG. 10 is a diagram of specific Western-blot detection according to an example of this disclosure.

FIG. 11 is a gel electrophoresis diagram of total RNA samples according to an example of this disclosure.

FIG. 12 is an electrophoretic analysis diagram of PCR amplification products of camel antibody fragments according to an example of this disclosure.

In this figure, M is Marker, 1 is a positive control, and 2 is PD-L1.

FIG. 12-A shows that the four samples all have two bands, wherein the main band has a molecular weight of about 600 bp, and there is a non-target band at 900 bp (this band should be an amplified fragment of a traditional antibody). A sufficient amount of PCR products is subjected to electrophoresis, and the main band of 600 bp is recovered by gel extraction, and used as a template for subsequent PCR to amplify VHH using VHH-back and PMCF primers.

FIG. 12-B shows PCR amplification leads to the obtaining of a target band with a molecular weight as expected (about 400 bp).

FIG. 13 is a diagram of vector self-ligation detection according to an example of this disclosure.

FIG. 14 is a diagram of colony counts of ligation-resistant lab test plates according to an example of this disclosure.

FIG. 15 is a diagram of storage capacity determination of the VHH library according to an example of this disclosure.

FIG. 16 is an analysis diagram of agarose gel electrophoresis of colony PCR products according to an example of this disclosure.

FIG. 17 is a diagram of detection results of target protein PD-L1 by SDS-PAGE according to an example of this disclosure.

FIG. 18 is a diagram of detection results of biotin labeling efficiency according to an example of this disclosure.

FIG. 19 is a schematic diagram showing the cytotoxicity of PD-L1 nanobody BHK-21 cells, sheep kidney cells, and MDBK cells according to an example of this disclosure. In this figure, the abscissa is the treatment of different concentrations of PD-L1 (μg/ml), and the ordinate is the OD value at 492 nm. BHK-21 is the experimental group of BHK-21 cells. Kidney is the experimental group of sheep kidney cells, MDBK is the experimental group of MDBK cells, and the data are expressed as mean±SD.

FIG. 20 is a schematic diagram showing the NO enhancement effect of PD-L1 on primary cells after the induction of mouse bone marrow stem cells according to an example of this disclosure. In this figure, negative is a negative control of the kit; PBS is a negative control with PBS addition, PD-L1 is the experimental group with addition of PD-L1 nanobodies.

FIG. 21 is a schematic diagram showing expression of PD-L1 receptor and PD-L1 using MC-38 cells according to an example of this disclosure.

FIG. 22 is a schematic diagram showing the results of flow cytometric detection of nanobodies and MC-38 cells according to an example of this disclosure.

### DESCRIPTION OF THE EMBODIMENTS

In order to make the objects, technical solutions and advantages of this disclosure clearer, this disclosure is further described in detail in combination with the examples below. It should be understood that the specific examples described herein are only used to explain this disclosure, and are not intended to limit this disclosure.

The application principle of this disclosure is described in detail below with reference to the drawings.

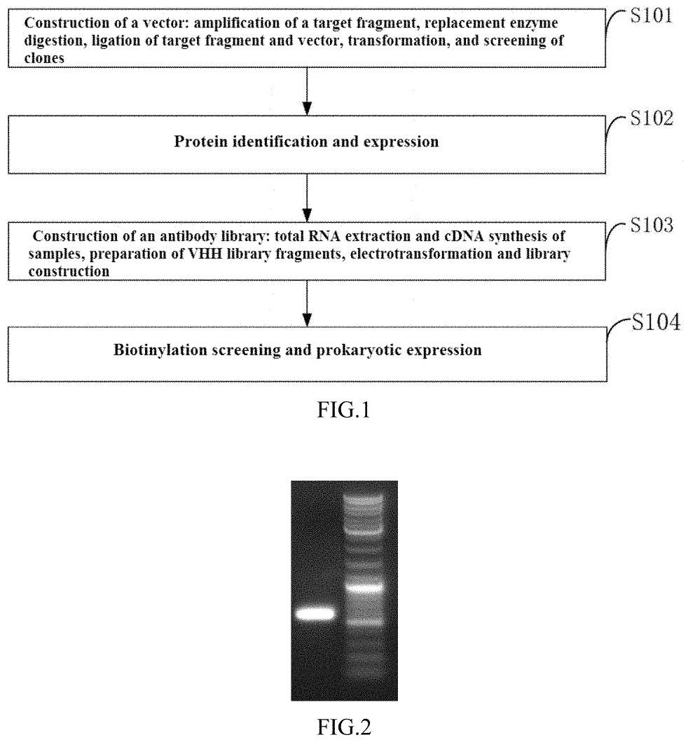

As shown in FIG. 1, a method is provided for preparing PD-L1 nanobodies from an alpaca immunized with PD-L1 antigens expressed in mammalian cells according to an example of this disclosure, wherein the PD-L1 antigen is transiently transfected and expressed in mammalian cells; the immunized animal is alpaca; screening is carried out by biotinylation; and the nanobody fragments obtained by screening have their own unique gene sequences.

Specifically, the following steps are included:

S101: construction of a vector: amplification of a target fragment, replacement enzyme digestion, ligation of target fragment and vector, transformation, and screening of clones.

S102: protein identification and expression.

S103: construction of an antibody library: total RNA extraction and cDNA synthesis of samples, preparation of VHH library fragments, electrotransformation and library construction.

S104: biotinylation screening and prokaryotic expression.

Another object of this disclosure is to provide a PD-L1 nanobody screened by the screening and identification method of the PD-L1 nanobody, wherein the DNA sequence of the PD-L1 nanobody is:

SEQ ID NO: 1
CGGGGCGGGAACATTTCCAAGCTTAAGGAGACAGTACATATGAAATACCT
ATTGCCTACGGCAGCCGCTGGATTGTTATTACTCGCGGCCCAGCCGGCCA
TGGCCCAGGTGCAGCTGCAGGAGTCTGGGGGAGGCTTGGTGCAGGCTGGG
GGCTCTCTGAGACTCTCCTGTGCAGCCTCTGGACGCACCTTCAGAAACGA
TGTCATGGCCTGGTTCCGCCAGATTCCAGGGAAGGAGCGTGAGTTTGTTG
CGGTGATTGCCTACGATGCGGCTGACACAGACTACGCAGACTCCGTGAAG
GGCCGATTCATCATCTCCAGAGACAACGCCAAGAACACGATATATTTGCA
AATGAACACCCTGAAACCTGAGGACACGGCCGTTTATTACTGTGCAGCCG
ACAAGGACAGAATGTACGGTAGTAGGCACTGGCCGGAATATGAGTATGAC
TACTGGGGCCAGGGGACCCAGGTCACCGTCTCCTCAGCGGCCGCATACCC
GTACGACGTTCCGGACTACGGTTCCCACCACCATCACCATCACTAGACTG
TTGAAAGTTGTTTAGCAAAACCTCATACAGAAAATTCATTTACTAACGTC
TGGAAAGACGACAAAACTTTAGATCGTTACGCTAACTATGAGGGCTGTCT
GTGGAATGCTACAGGCGTTGTCGTTTGTACTGGTGACGAAACTCAGTGTT
ACGGTACATGGGTTCCTATTGGGCTTGCTATCCCTGAAAATGAGGGTGGT
GGCTCTGAGGGTGGCGGTTCTGAGGGTGGCGGTTCTGAGGGTGGCGGTAC
TAAACCTCCTGAGTACGGTGATACACCTATTCCGGGCTATACTTATATCA
ACCCTCTCGACAGCACTTATCCGCCTGGTACTGGAGCAAAACCCCGCTAA
TCCTAAATCCTTCTCTTGGAGGAGTCTCAGCCTCTTAATACTTTCATGTT
TCAGAATAATAGGTCCGAATAAGGCAGGGTGCATAAGCTGTTTATACGGG
ACTGTTACTCAAGGCACTGACCCGATTAAAGTTAGTAACAGTACACTCCC
GTGAATCATACGAAGCATGGTAGGACGCTTAACTGGGACAGGAAAAGTC.

In step S101, the amplification of the target fragment according to an example of this disclosure specifically comprises:

(1) Designing Synthetic Primers:

PD-L1 upstream primer

| | |
|---|---|
| 5′-GACACGAATTCGCCACC-3′. | SEQ ID NO: 2 |

PD-L1 downstream primer

| | |
|---|---|
| 5′-GTGTCAAGCTTTCACTTATCATCA-3′. | SEQ ID NO: 3 |

Amplifying a sufficient amount of target product (PD-L1 Uniprot: Q9NZQ7) by PCR.

(2) Using a pfu high-temperature polymerase for PCR reaction.

In step S101, the amount of components of PCR according to an example of this disclosure is as follows:

The primer concentration is 1 OD soluble in 400 μl of ddH₂O.

| Reaction system | 50 | μl |
|---|---|---|
| Primer mix | (1/34) 0.4 μl × 13.6 | |
| | 8 μl in total | |
| 10 × pfu Buffer | 5 | μl |
| Upstream and downstream | 2 | μl each |
| primers of each segment | | |
| Pfu | 0.4 | μl (5 U/μl) |
| ddH₂O | To 50 | μl |

In step S101, the specific steps of PCR amplification of target fragment according to an example of this disclosure include:

(1) A First Round PCR Procedure:

| | | |
|---|---|---|
| 95° C. 3 min | | |
| 95° C. 22 sec | | |
| 50° C. 20 sec | {close oversize brace} | 18 cycles |
| 72° C. 40 sec | | |
| 72° C. 5 min | | |

The above is a first round PCR reaction system, and the first round PCR product is used as the template for a second round PCR.

(2) A Second Round PCR Reaction System:

The amount of components of PCR is as follows: The primer concentration is 1 OD soluble in 400 μl of ddH₂O.

| | | |
|---|---|---|
| Upstream primer-1 | 2 | μl |
| Downstream primer-34 | 2 | μl |
| First round PCR product | 1 | μl |
| dNTP | 1 | μl (25 mM each) |
| 10 × pfu Buffer | 5 | μl |
| Pfu | 0.4 | μl (5 U/μl) |
| ddH₂O | To 50 | μl |

(3) A Second Round PCR Procedure:

| | | |
|---|---|---|
| 95° C. 3 min | | |
| 95° C. 22 sec | | |
| 55° C. 20 sec | {close oversize brace} | 22 cycles |
| 72° C. 45 sec | | |
| 72° C. 5 min | | |

The second round of PCR is subjected to agarose gel electrophoresis to recover purified fragments for enzyme digestion.

In step S101, the above PCR product is digested by replacement enzyme digestion according to an example of this disclosure.

| | | |
|---|---|---|
| PCR product fragment digestion system | 50 | μl |
| Recovered purified fragment | 1 | μg (20 μl) |
| 10 × FD Buffer | 5 | μl |
| EcoRI | 1 | μl (10 U/μl) |
| HindIII | 1 | μl (10 U/μl) |
| ddH₂O | 23 | μl U/μl |

The above system is reacted in a 37° C. constant temperature water bath for 2 h.

Enzyme Digestion System of Vector:

| | | |
|---|---|---|
| PCDNA3.1+ | 1 | μg |
| 10 × FD Buffer | 5 | μl |
| EcoRI | 1 | μl (10 U/μl) |
| HindIII | 1 | μl (10 U/μl) |
| ddH₂O | 42 | μl |

The above system is reacted in a 37° C. constant temperature water bath for 2 h.

The digested vectors and fragments are recovered.

In step S101, the recovered purified target DNA fragments are ligated to vectors according to an example of this disclosure.

Ligation system: 20 μl.

Enzyme-digested target fragment: 8 μl.

Enzyme-digested vector PCDNA3.1+4 ul. μl

10×T4 DNAligase Buffer 2 μl.

T4 DNAligase 1 μl (5 U/μl ).

ddH₂O supplemented to 20 μl.

The above ligation mix solution is placed in a PCR machine at 22° C. for 1 h.

In step S101, transformation and screening of clones are carried out according to an example of this disclosure:

The above ligation solution is transferred into oneshort competent cells to detect and screen positive clones for sequencing.

The application principle of this disclosure is further described below in combination with specific examples.

### EXAMPLE 1

### Vector Construction According to an Example of This Disclosure

1. Amplification of the target fragment is specifically as follows:

(1) 34 synthetic primers are designed to obtain a sufficient amount of the target product by PCR amplification.

(2) The PCR reaction uses a pfu high-temperature polymerase.

The amount of components of PCR: (The primer concentration is 1 OD soluble in 400 μl of ddH₂O).

Reaction system: 50 μl. Primer mix, (1/34) 0.4 μl×13.6 a total of 8 μl. 10×pfu Buffer, 5 μl. Each segment of upstream and downstream primers, 2 μl each. Pfu, 0.4 μl (5 U/μl). ddH₂O, added to 50 μl, respectively.

The specific steps of PCR amplification of the target fragment include:

(1) A First Round PCR Procedure:

| | | |
|---|---|---|
| 95° C. 3 min | | |
| 95° C. 22 sec | | |
| 50° C. 20 sec | {close oversize brace} | 18 cycles |
| 72° C. 40 sec | | |
| 72° C. 5 min | | |

The above is a first round PCR reaction system, and the first round PCR product is used as the template for a second round PCR.

(2) A Second Round PCR Reaction System:

The amount of components of PCR is as follows: The primer concentration is 1 OD soluble in 400 μl of ddH₂O.

| | | |
|---|---|---|
| Upstream primer-1 | 2 | μl |
| Downstream primer-34 | 2 | μl |
| First round PCR product | 1 | μl |
| dNTP | 1 | μl (25 mM each) |
| 10 × pfu Buffer | 5 | μl |
| Pfu | 0.4 | μl (5 U/μl) |
| ddH₂O | To 50 | μl |

(3) A Second Round PCR Procedure:

| | | |
|---|---|---|
| 95° C. 3 min | | |
| 95° C. 22 sec | | |
| 55° C. 20 sec | {close oversize brace} | 22 cycles |
| 72° C. 45 sec | | |
| 72° C. 5 min | | |

As shown in FIG. 2, an original diagram of PCR agarose gel electrophoresis is provided according to an example of this disclosure. Purified fragments are recovered for enzyme digestion.

2. Replacement enzyme digestion, the above PCR product is digested.

| | | |
|---|---|---|
| PCR product fragment digestion system | 50 | μl |
| Recovered purified fragment | 1 | μg (20 μl) |
| 10 × FD Buffer | 5 | μl |
| EcoRI | 1 | μl (10 U/μl) |
| HindIII | 1 | μl (10 U/μl) |
| ddH₂O | 23 | μl |

The above system is reacted in a 37° C. constant temperature water bath for 2 h.

Enzyme Digestion System of Vector:

| | | |
|---|---|---|
| PCDNA3.1+ | 1 | μg |
| 10 × FD Buffer | 5 | μl |
| EcoRI | 1 | μl (10 U/μl) |
| HindIII | 1 | μl (10 U/μl) |
| ddH₂O | 42 | μl |

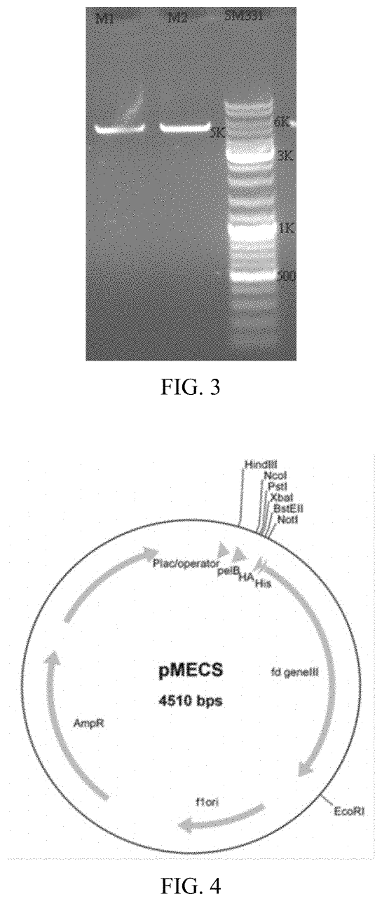

As shown in FIG. 3, an agarose gel electrophoresis diagram of vector PCDNA3.1+ after enzyme digestion is provided according to an example of this disclosure.

The above system is reacted in a 37° C. constant temperature water bath for 2 h.

The digested vectors and fragments are recovered.

3. Ligation of target fragments and vectors, the recovered purified target DNA fragments are ligated to vectors.

| | | |
|---|---|---|
| Ligation system | 20 | μl |
| Digested target fragment | 8 | μl |
| Digested vector PCDNA3.1+ | 4 | μl |
| 10 × T4 DNAligase Buffer | 2 | μl |
| T4 DNAligase | 1 | μl (5 U/μl) |
| ddH₂O | To 20 | μl |

4. Transformation and Screening of Clones:

The above ligation mix solution is placed in a PCR machine at 22° C. for 1 h.

### EXAMPLE 2

The construction of a nanobody library according to an example of this disclosure specifically includes:

1. Experimental Design

The M13 phage display system is selected to display the VHH antibody library, which consists of pMECS phagemid vector, E. coli TG1 and M13K07 helper phage. In the phagemid vector pMECS, the sequence before the Pst I restriction site is the coding sequence of the pelB secretion signal peptide and part of the amino acids in the first framework region of the antibody. The pelB signal peptide can guide the secretion of the subsequent polypeptide to the periplasmic cavity. Not I restriction site is followed by the coding sequence of HA and 6×His tag, which can be used for purification or detection of fusion protein. The sequence immediately following encodes the bacteriophage PIII capsid protein (shown in FIG. 4). There is an amber stop codon between the 6×His tag and the gene III sequence. In amber stop codon-suppressed strains (such as E. coli TG1), 10% to 20% of the amber stop codons can be translated into glutamic acid (Glu, or E), VHH and gene III protein fusion expression, when rescued with helper phage M13K07, VHH antibody is displayed at the N-terminus of PIII protein in the tail of phage.

As shown in FIG. 4, the sequence according to an example of this disclosure encodes a phage PIII capsid protein.

Therefore, the total RNA of the sample is first extracted and reversely transcribed into cDNA, and then the CAL-leader and CAL-CH2 primer pairs are used to amplify the camel antibody fragments. The ˜600 bp band of the above PCR is recovered by gel extraction, and used as a template for subsequent PCR to amplify the VHH gene fragment with VHH-back and PMCF primers. Not I restriction site is introduced at the 3′end of the VHH gene fragment (the VHH fragment has Pst I restriction site at the 5′end), and the fragment is inserted into the pMECS phagemid vector by enzyme digestion and ligation reaction, and subsequently transformed into E. coli TG1 to construct an M13 single-chain filamentous phage display camel nanobody immune library.

2. Experimental Materials

(1) Lymphocyte samples of camel peripheral blood are collected from camel peripheral blood.

(2) cDNA synthesis, PCR amplification, restriction endonucleases, T4 DNA ligases and other kits and tool enzymes are mainly purchased from companies such as Thermo Scientific and New England Biolabs.

(3) Experimental materials such as pMECS, E. coli TG1, Helper phage M13K07 and the like are preserved by the zoonotic laboratory.

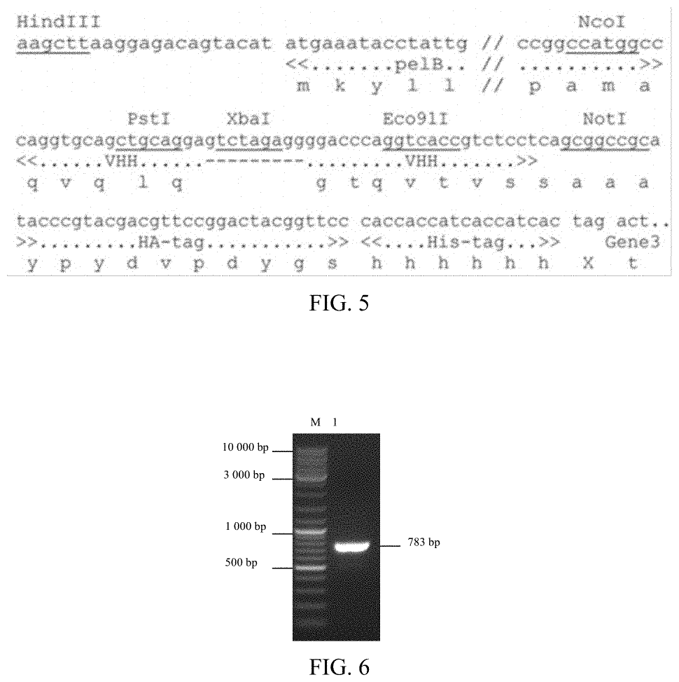

As shown in FIG. 5, it is a diagram of phagemid pMECS plasmids according to an example of this disclosure.

2. Experimental Results

(1) Sample Total RNA Extraction and cDNA Synthesis

Trizol reagents are used to extract total RNA from the peripheral blood lymphocyte samples of camel, and the quality of total RNA is detected by agarose gel electrophoresis.

As shown in FIG. 11, it is a gel electrophoresis diagram of total RNA samples according to an example of this disclosure.

In this figure, M is DL2 000 DNA marker.

There is a very slight degradation in total RNA samples. 38S, 18S and 5S rRNA bands are all clearly visible; moreover, the 28S band brightness is greater than 18S, indicating that the RNA integrity is good. The concentration of RNA samples is measured by Nanodrop, and the results show that the concentration and purity of the RNA samples meet the requirements (Table 1). Reverse transcription is used to synthesize cDNA using 10 μg of total RNA as a template.

TABLE 1
Total RNA sample concentration and purity

| Sample | Concentration (ng/μl) | OD₂₆₀/OD₃₄₀ |
|---|---|---|
| PD-L1 | 740.9 | 2.04 |

(2) Preparation of VHH Library Fragment

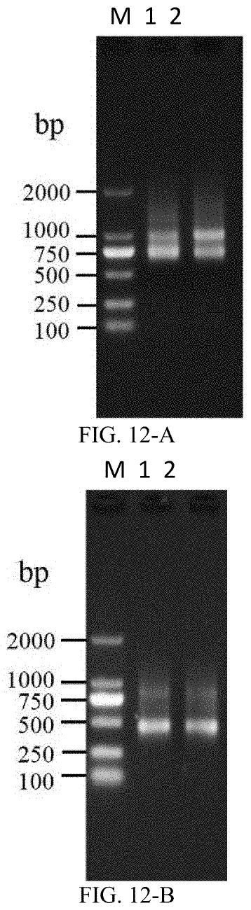

Camel antibody fragments are amplified with primers CAL-leader and CAL-CH2 using the above cDNA as the template, respectively, and an appropriate amount of PCR products are detected by 1% agarose gel electrophoresis. The results are shown in FIG. 12-A: Samples all have two bands, wherein the main band has a molecular weight of about 600 bp, and there is a non-target band at 900 bp (this band should be an amplified fragment of a traditional antibody). A sufficient amount of PCR products is subjected to electrophoresis, and the main band of 600 bp is recovered by gel extraction, and used as a template for subsequent PCR to amplify VHH using VHH-back and PMCF primers. The results are as shown in FIG. 12-B: PCR amplification leads to the obtaining of a target band with a molecular weight as expected (about 400 bp).

As shown in FIG. 12, it is an electrophoretic analysis diagram of PCR amplification products of camel antibody fragments according to an example of this disclosure.

A, Camel antibody fragment 1^{st} PCR amplification. B, Camel nanobody VHH fragment 2^{nd} PCR amplification.

(3) Electrotransformation and Library Construction

### EXAMPLE 3

Protein identification and expression according to an example of this disclosure specifically include:

(1) Construction of Mammalian Cell Expression Vector

1. Amplify and extract vector plasmids containing a gene of interest.

2. Subclone into eukaryotic expression vector pcDNA3.1.

3. Sequence and verify the accuracy of plasmid construction.

4. Obtain plasmid pcDNA3.1 by extraction.

(2) Mammalian Cell Culture, Protein Expression and Purification Test

1. Cell Lines and Materials

Cell line: HEK293 cells.

Media: DMEM (10% serum), DMEM (no serum).

Culture utensils: 10 cm dish or 15 cm dish

2. Transfection of HEK293 Cells (10 cm Dish)

(1) 24 h before transfection, the plate is charged with a total amount of cells in a 4-5 106/10 cm culture dish. When cell growth is in good condition and the adherent cell density reaches 50-80%, transfection can be performed.

(2) 5 μg of plasmid DNA to be transfected is added to a 1.5 ml centrifuge tube and evenly mixed.

(3) 10 μl liposomes is added to 500 μl DMEM culture solution; the above DNA is added, gently mixed, and incubated at RT for 30 min.

(4) The liquid containing DNA and liposomes is carefully added to a culture dish, evenly dispersed, and placed in a 37° C. 5% CO₂ incubator for 72 h.

3. Observation and Collection of Cells

(1) 72 h after transfection, the cell culture medium is carefully pipetted, and the adherent cells are rinsed once with pre-chilled PBS.

(2) Cell precipitates are collected by centrifugation.

4. Lysis of Cells

(1) Cell Lysis Buffer: 50 mM Tris (PH 8.0), 300 mM NaCl, 1% Triton X-100, 1 mM DTT, 5% glycerol, is added.

(2) 200 W ice bath ultrasound is performed for 10 min.

(3) 16 000 rpm, 20 min, 4° C., the lysis supernatant is collected.

5. Flag Tag Purification

(1) A Flag filler is used, and a 1 ml column is used for purification.

(2) The column is equilibrated 10 CV beforehand with Binding Buffer: (50 mM Tris (pH 8.0), 300 mM NaCl, 0.1% Triton X-100, 1 mM DTT, 5% glycerol).

(3) The lysed cell supernatant is added to the equilibrated column.

(4) After loading the sample, the column is washed with Binding Buffer.

(5) The column is washed 5-10 CV with Wash Buffer: (50 mM Tris (pH 8.0), 500 mM NaCl, 1 mM DTT, 5% glycerol), and non-specific adsorption impurities are washed off, if adhered.

(6) The target protein is eluted with Elution Buffer: (50 mM Tris (pH 8.0), 150 mM NaCl, 150 μg/μl Flagpeptide, 1 mM DTT, 10% glycerol), and collected.

The disclosure pertains to the technical field of virtual reality, and specifically pertains to an interaction control method and an interaction control device for virtual reality.

6. Western-Blot Detection

(1) Solution Preparation:

Transfer buffer: 0.025 M Tris base, 0.192 M glycine, 30% methanol 10×TBST: 250 mM Tris-HCl (pH 8.0), 1.25 M NaCl, 0.5% Tween 20

Blocking buffer: 1×TBST, 3% skimmed milk powder

Washing solution: 1×TBST

(2) Experimental Procedure:

A. Membrane preparation: The PVDF membrane is cut into strips and immersed in methanol, shaken at room temperature on a shaker for 1 min; after removing methanol, 1×TBST is added.

B. Membrane Transfer:

i. SDS-PAGE electrophoresis.

ii. Electrically transfer to PVDF membrane by sandwich method.

iii. Pre-wet sponge and filter paper in the transfer buffer.

iv. Transfer with 300 mA for 80 min, and block with the blocking buffer for 1 h at room temperature or overnight at 4° C.

C. Antibody Detection:

i. Dilute the primary antibody anti-Flag tag according to instructions, and incubate overnight at 4° C.

ii. Wash 3 times with the washing solution, 5 min each time.

iii. Dilute the secondary antibody with the blocking buffer and incubate at room temperature for 1 h.

iv. Wash 3 times with the washing solution, 5 min each time.

v. Detect through TMB color development.

(3) Protein Identification Results:

Notes about protein electrophoresis molecular weight and electrophoresis band:

(1) Since the specific amino acid composition and arrangement of each protein are different, even proteins with very close molecular weights still behave differently on SDS-PAGE gels. The protein molecular weight marker is a reference for molecular weight. On SDS-PAGE electrophoresis, the target protein may completely match the theoretical molecular weight, or it may be higher or lower.

(2) Secreted protein samples are modified with glycosylation, and the molecular weight is usually higher than the theoretical molecular weight. In addition, the glycosylation modification process of the protein is not completely uniform. It is often seen that the protein bands of target protein are dispersed and include multiple bands that reside close to each other. These are typical electrophoretic performances of secreted proteins.

This disclosure is further described below in combination with Experiment 1.

Experimental Method:

Protein expression and alpaca immunization: The experiment uses the PCR method to amplify PD-L 1 extracellular domain sequences, constructs a recombinant pcDNA3.1 plasmids, transfects the plasmids into HEK293 cell line to express the target protein, and subjects the target protein to SDS-PAGE and Western-blot detection, analyzes protein properties by ProtParam software, and predicts protein structure by SWISS-MODEL software. The results show: The PCR method has successfully amplified the target band, and 1% agarose gel electrophoresis shows that the enzyme digestion is successful. 10% SDS-PAGE detection shows that the target protein is successfully expressed. Western-blot detection proves that the protein is specific. The Nanobody library is successfully constructed. Highly specific nanobodies are successfully screened.

1 Materials and Methods

1.1 Material

1.1.1 Plasmids and Strains

The pcDNA3.1 plasmids, HEK293 cells and HEK293 competent cells are all preserved by the laboratory jointly established for Xinjiang Ethnic and Local High-risk Diseases.

1.1.2 Main Biochemical Reagents

Fetal bovine serum and DMEM medium are purchased from Gibco company. EcoR I endonucleases, HindIII endonucleases, mouse anti-His monoclonal IgG antibodies, and horseradish peroxidase (HRP)-labeled rabbit anti-mouse IgG are all purchased from Sangon Biotech (Shanghai) Co., Ltd. Nickel column is purchased from GE company. TMB color developing solution is purchased from Beijing Zhongshan Jinqiao Biotechnology Co., Ltd. SM331 GeneRuler DNA Ladder Mix (Thermo Scientific). (The rest of the reagents are produced by Sangon company). The enzyme used is NdeI, XhoI enzyme produced by Thermo Scientific and the corresponding FD Buffer. The electrophoresis instrument is DYY85 type from Beijing Liuyi Instrument Factory. The PCR product purification uses a PCR purification kit manufactured by Sangon. The ligase used for 10×T4 DNAligase Buffer is produced by Thermo Thermo Scientific.

1.2 Method:

1.2.1 Preparation of PD-L1 Immunogen:

The target sequence is obtained using the Uniprot database; primers are designed using Primer Premier 5.0 software, and synthesized by Sangon Biotech (Shanghai) Co., Ltd. Synthesized by Sangon Biotech (Shanghai) Co., Ltd., the synthesized fragment is used as the PCR template.

| Primer name | Primer sequence |
|---|---|
| PD-L1 | 5′-GACACGAATTCGCCACC-3′ |
| upstream primer | (SEQ ID NO: 2) |
| PD-L1 | 5′-GTGTCAAGCTTTCACTTATCATCA-3′ |
| downstream primer | (SEQ ID NO: 3) |

The amount of components of PCR: (The primer concentration is 1 OD soluble in 400 μl of ddH₂O).

PCR system

| Component | Volume | |
|---|---|---|
| Upstream primer | 2 | μl |
| Downstream primer | 2 | μl |
| Target gene | 3 | μl |
| dNTP | 1 | μl (25 mM each) |
| 10 × pfu Buffer | 5 | μl |
| Pfu | 0.4 | μl (5 U/μl) |
| ddH2O | To 50 | μl |

PCR procedures for PD-L1 target fragments, TIM-3 target fragments and CTLA-4 target fragments:

PCR procedure

| Temperature | Time | | |
|---|---|---|---|
| 95° C. | 3 min | | |
| 95° C. | 22 sec | | |
| 55° C. | 20 sec | {close oversize brace} | 22 cycles |
| 72° C. | 45 sec | | |
| 72° C. | 5 min | | |

After the completion of PCR, 1% agarose gel electrophoresis is performed, and the purified fragments are recovered for enzyme digestion.

1.2.2 Enzyme Digestion and Identification

The above PCR products are digested with 50 μl of PCR product fragment digestion system.

PCR product fragment digestion system 50 μl

| Component | Volume | |
|---|---|---|
| Purified recovered fragments | 1 | μl (20 μl) |
| 10 × FD Buffer | 5 | μl |
| EcoRI | 1 | μl (10 U/μl) |
| HindIII | 1 | μl (10 U/μl) |
| ddH₂O | 23 | μl |

The above system is placed into a 37° C. constant temperature water bath for 2 h.

1.2.3 Vector Digestion System:

Vector digestion system

| Component | Volume | |
|---|---|---|
| PCDNA3.1+ | 1 | μg |
| 10 × FD Buffer | 5 | μl |
| EcoRI | 1 | μl (10 U/μl) |
| HindIII | 1 | μl (10 U/μl) |
| ddH₂O | 42 | μl |

The above system is placed into a 37° C. constant temperature water bath for 2 h to recover the digested vectors and fragments.

Ligation of Target Fragment and Vector

The recovered purified DNA fragment and vector are ligated.

Ligation system: 20 μl

| | | |
|---|---|---|
| Digested target fragment | 8 | μl |
| Digested vector PCDNA3.1+ | 4 | μl |
| 10 × T4 DNAligase Buffer | 2 | μl |
| T4 DNAligase | 1 | μl (5 U/μl) |
| ddH₂O | To 20 | μl |

The ligation mix solution is placed in the PCR instrument at 22° C. for 1 h. The above ligation solution is transferred into HEK293 competent cells by 42° C. heat shock method; positive clones are detected and screened to extract recombinant plasmids.

1.3 SDS-PAGE Detection

The plate is charged 24 h before transfection of HEK293 cells, and transfection is performed when cell growth is in good condition and the adherent cell density reaches 50-80%. 10 μl liposomes are added to 500 μl DMEM medium; 5 μg recombinant plasmids are added, gently mixed, and incubated at room temperature for 30 min. The liquid containing DNA and liposomes is carefully added to a culture dish, evenly dispersed, and placed in a 37° C., 5% CO₂ incubator for 72 h. The cell culture medium is carefully pipetted, and the cells are collected by centrifugation; Cell Lysis Buffer is added, and sonicated in a 200 W ice bath for 10 min. 16 000 r/min for 20 min. The lysate supernatant is collected and subjected to 10% SDS-PAGE electrophoresis. After the determination of protein expression, the protein is purified using a nickel column.

1.4 Specific Western-Blot Detection

The PVDF membrane is cut into strips and immersed in methanol, and incubated in a shaker at room temperature for 1 min. After removing methanol, 1×TBST is added for SDS-PAGE electrophoresis, and the protein is transferred to PVDF membrane by sandwich method. After pre-wetting the filter paper by immersing in the transfer buffer and, transferring is performed at 300 mA for 80 min. Blocking is performed with blocking buffer for 1 h at room temperature. The primary antibody is diluted 3 000 times and incubated at 4° C. overnight. Washing is performed 3 times with PBST for 5 min each time. Then the secondary antibody is diluted 5 000 times with blocking buffer and incubated for 1 h at room temperature. Washing is performed 3 times with PBST for 5 min each time, and color is developed using TMB color developing solution. PD-L1 primary antibody is Anti-PD-L1 (ABM4E54) (mouse monoclonal antibody (ABM4E54) to PD-L1, Anti-PD-L1 antibody (ABM4E54) ab210931); secondary antibody is goat polyclonal antibody secondary antibody to mouse IgG-H&L (HRP) antibody (abcam company ab6789).

1.5 Immunization of Alpaca

The first immunization is carried out by subcutaneous and intradermal injection with Freund's complete adjuvant 0.5 ml+protein 0.5 ml emulsification. Freund's incomplete adjuvant 0.25 ml+protein 0.25 ml is evenly emulsified, and the alpaca is immunized subcutaneously at 28 days (2^{nd} immunity), 49 days (3^{rd} immunity), and 70 days (4^{th} immunity). Freund's incomplete adjuvant 0.125 ml+protein 0.125 ml is evenly emulsified, and the alpaca is immunized at 91 days (5th immunity), 112 days (6^{th} immunity), 133 days (7^{th} immunity). On day 144, lymphocytes are isolated.

Immunization scheme

| | | | | | | | | Lymphocyte |
|---|---|---|---|---|---|---|---|---|
| Scheme | 1^{st} | 2^{nd} | 3^{rd} | 4^{th} | 5^{th} | 6^{th} | 7^{th} | isolation |
| Immunization | Day | Day | Day | Day | Day | Day | Day | Day 144 |
| time | 0 | 28 | 49 | 70 | 91 | 112 | 133 | PD-L1 |

| | | | |
|---|---|---|---|
| Immunization | 1 ml | 0.5 ml | 0.25 ml |
| dose | | | |

| | | |
|---|---|---|
| Adjuvant | Freund's | Freund's incomplete adjuvant |
| | complete | |
| | adjuvant | |
| Immunization | Subcutaneous, | Subcutaneous |
| manner | intradermal | |

Blood collection time

| | |
|---|---|
| Day 0 | Day 7 |
| Day 28 | Day 35 |
| Day 49 | Day 56 |
| Day 70 | Day 77 |
| Day 91 | Day 98 |
| Day 112 | Day 119 |
| Day 133 | Day 140 |

1.5.1 ELISA Test Steps

Antigens are 200 ng/well and coated overnight at 4 degrees. The plate is washed once with PBST (0.1%), 1×blocker 300 μl/well, and blocked at 37 degrees for 2 h. The plate is washed once with PBST (0.1%); the antiserum is diluted with a 0.5×blocker gradient; 100 μl/well is added to the plate, 37 degrees for 1 h. The plate is washed 3 times with PBST (0.1%), the anti-alpaca secondary antibody is diluted 1:15 000 with 0.5×blocker; and 100 μl/well is added to the plate at 37 degrees for 1 h. The plate is washed 3 times with PBST (0.1%), and then washed 3 times with PBS; 100 μl/well TMB color development for about 20 min, and terminated with 50 μl/well 2 M H₂SO₄. Microplate reader OD450-OD630 nm reading.

1.5.2 Lymphocyte Isolation:

The lymphocyte separation solution is preheated to 22° C. 200 ml peripheral blood is collected from each alpaca using a heparin sodium anticoagulation tube. Whole blood is diluted with an equal volume of tissue diluent. An equal volume of separation solution is added to the centrifuge tube. Centrifugation is performed at room temperature, horizontal rotor 1 000 g for 30 min. The white membrane layer is pipetted; 10 mL PBS washing solution is added to wash the white membrane layer cells. 250 g, centrifuged for 10 min. The supernatant is discarded, and the cells are resuspended in 5 ml PBS, 250 g, centrifuged for 10 min. The supernatant is discarded, the cells are resuspended in 5 ml PBS, 250 g, centrifuged for 10 min. The supernatant is discarded, and the cells are resuspended using Trizol.

This disclosure is further described in combination with the construction of the camel nanobody immune library in Experiment 1.

Camel peripheral blood lymphocyte total RNA is extracted and reversely transcribed into cDNA. immune library. Camel antibody primer pairs are used to amplify four VHH library fragments, respectively. The four VHH library fragments are inserted into pMECS phagemid vectors, respectively, and transformed into E. coli TG1 to construct a phage display antibody immune library with a storage capacity of >10⁹. Twenty clones are randomly picked from the library for sequencing and analysis to ensure that more than 99% of the clones in the library contain the target insertion sequence.

1. Experimental Materials

cDNA synthesis, PCR amplification, restriction endonucleases, T4 DNA ligases and other kits and tool enzymes are mainly purchased from companies such as Thermo Scientific and New England Biolabs. Experimental materials such as pMECS, E. coli TG1, Helper phage M13K07 and the like are preserved in the laboratory

2. Experimental method includes:

Sample Total RNA Extraction and cDNA Synthesis:

Trizol is used to extract the total RNA of camel peripheral blood lymphocyte samples, and the quality of the total RNA is detected by agarose gel electrophoresis.

Preparation of VHH Library Fragments:

Camel antibody fragments are amplified with primers CAL-leader and CAL-CH2 using cDNA as a template, respectively, and an appropriate amount of PCR products is subjected to 1% agarose gel electrophoresis detection.

| Primer CAL-leader sequence: | |
|---|---|
| GTCCTGGCTGCTCTTCTACAAGG. | (SEQ ID NO: 4) |
| Primer CA-CH2 sequence: | |
| GGTACGTGCTGTTGAACTGTTCC. | (SEQ ID NO: 5) |

PCR System:

A pfu high-fidelity DNA polymerase (TransStart FastPfu DNA Polymerase, AP221-01) is used.

| Component | Volume | Final Concentration | |
|---|---|---|---|
| 5 × pfu Buffer | 10 μl | 1× | |
| dNTPs (2.5 mM each) | 4 μl | 0.2 | mM |
| CAL-leader (10 μM) | 1 μl | 0.2 | μM |
| CAL-CH2 (10 μM) | 1 μl | 0.2 | μM |
| cDNA | 2 μl | | |
| pfu DNA polymerase | 1 μl | 2.5 | units |
| ddH₂O | 31 μl | | |

PCR Procedure

| Number of cycles | | Temperature | Time |
|---|---|---|---|
| 1 cycle | | 95° C. | 2 min |
| | | 95° C. | 30 s |
| 30 cycles | {open oversize brace} | 56° C. | 30 s |
| | | 72° C. | 1 min |

| | | |
|---|---|---|
| 1 cycle | 72° C. | 5 min |
| 1 cycle | 4° C. | ∞ |

Ligation and Transformation Pretests:

Before the formal establishment of the library, the quality of phagemid vectors, ligation efficiency of vectors and VHH library fragments are tested and evaluated by ligation and transformation pretests. VHH fragments digested with Pst I/Not I and phagemid vectors pMECS digested with the same Pst I/Not I are subjected to a ligation reaction at different ratios using T4 DNA ligases (vectors used in each group are in the same amount), and then transformed into E. coli TG1 chemically competent cells. The coated samples have two bands, wherein the main band has a molecular weight of about 600 bp, and there is a non-target band at 900 bp, which band should be an amplified fragment of a traditional antibody. A sufficient amount of PCR products is subjected to electrophoresis, and the main band of 600 bp is recovered by gel extraction, and used as a template for subsequent PCR to amplify VHH using VHH-back and PMCF primers. PCR amplification leads to the obtaining of a target band with a molecular weight as expected, about 400 bp.

Primer VHH-back sequence:
(SEQ ID NO: 6)
GATGTGCAGCTGCAGGAGTCTGGRGGAGG.
Primer PMCF sequence:
(SEQ ID NO: 7)
CTAGTGCGGCCGCTGAGGAGACGGTGACCTGGGT.

PCR System:

| Component | Volume | | Final Concentration | |
|---|---|---|---|---|
| 5 × pfu Buffer | 10 | μl | 1× | |
| dNTPs (2.5 mM each) | 4 | μl | 0.2 | mM |
| PMCF (10 μM) | 1 | μl | 0.2 | μM |
| VHH-back (10 μM) | 1 | μl | 0.2 | μM |
| Template | X | μl | 50 ng/50 μl System | |
| Pfu DNA polymerase | 1 | μl | 2.5 | units |

| | |
|---|---|
| ddH₂O | To 50 μl |

PCR Procedure:

| Number of cycles | | Temperature | Time |
|---|---|---|---|
| 1 cycle | | 95° C. | 2 min |
| | | 95° C. | 30 s |
| 25 cycles | {open oversize brace} | 58° C. | 30 s |
| | | 72° C. | 1 min |

| | | |
|---|---|---|
| 1 cycle | 72° C. | 5 min |
| 1 cycle | 4° C. | ∞ |

Ampicillin-resistant plates is used for colony counting.

Electrotransformation and Library Construction:

In the ligation experiment of formal library construction, according to the optimal ratio obtained by ligation and transformation pretests, vectors are ligated with four VHH fragments. The purified ligation products are electrotransformed into E. coli TG1 to obtain 15 ml of transformation product. 10 μl (i.e., 10⁻² ml) is taken and subjected to a series of 10-fold gradient dilution, and three gradients, 10⁻⁴, 10⁻⁵ and 10⁻⁶, are used for counting on ampicillin-resistant plates to evaluate the library capacity, the library capacity=number of clones×dilution multiple×total volume (ml) of transformation products. The remaining transformation products are coated on 15 Amp-resistant plates with a diameter of 15 cm, cultured overnight, scraped off from each plate the next day, mixed evenly, to which 20% final concentration glycerol is added, packed in aliquots and cryopreserved at −80° C.

Electrotransformation Experiment Scheme:

1) E. coli TG1 electrotransformation competent cells are prepared.

2) Purified ligation products are added to an appropriate amount of TG1 competent cells, evenly mixed, and distributed into 0.2 cm electroporation cuvettes.

3) Electrotransformation is carried out using an electrotransformation instrument under transformation conditions recommended by BIORAD: 2.5 kV, 25 μF, 200Ω.

4) 2YT medium is added to the electroporation cuvettes, and the competent cells are resuspended, and rescued at 37° C. and 150 rpm for 30 min.

Quality Analysis of Immune Library:

Twenty monoclonals are randomly picked from the gradient dilution plate of each library, and colony PCR is performed using primers MP57 and PMCF. These clones are sequenced using primer MP57 (-TTATGCTTCCGGCTCGTATG-SEQ ID NO:8)

PCR System:

Taq DNA polymerase (2×Taq Plus MasterMix, CW2849M) is used.

| | | | Final |
|---|---|---|---|
| Component | Volume | | Concentration |
| 2 × Taq Plus MasterMix | 25 | μl | 1× |
| MP57 (10 μM) | 2 | μl | 0.4 μM |
| PMCF (10 μM) | 2 | μl | 0.4 μM |
| Template | 1 | μl | |
| ddH₂O | 20 | μl | |

PCR Procedure:

| Number of cycles | | Temperature | Time |
|---|---|---|---|
| 1 cycle | | 94° C. | 2 min |
| | | 94° C. | 30 s |
| 27 cycles | {open oversize brace} | 57° C. | 30 s |
| | | 72° C. | 1 min |

| | | |
|---|---|---|
| 1 cycle | 72° C. | 2 min |
| 1 cycle | 4° C. | ∞ |

This disclosure is further described below in combination with the experimental results in Experiment 1.

2 Results

2.1 PCR Amplification:

After PCR amplification, a target band with an expected size is obtained. The target band is clear as detected by 1% agarose gel electrophoresis with a size as expected, indicating successful amplification of the target band.

PD-L1 PCR amplification is shown in FIG. 6. In the figure, M represents DL-10 000 Marker. 1 denotes the target band. The lane is the whole length 783 bp of the amplified PCR target fragment.

2.2 Enzyme Digestion Identification

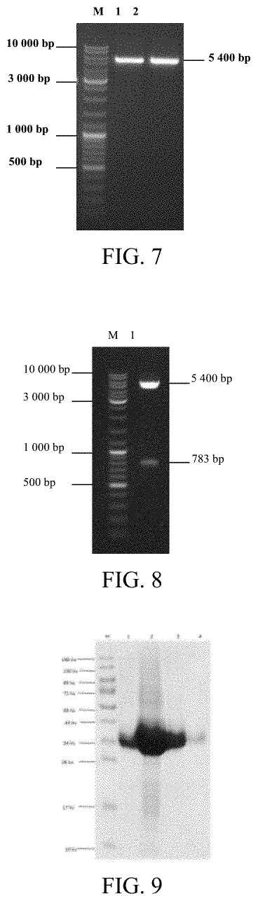

The pcDNA3.1 plasmid is detected by 1% agarose gel electrophoresis after enzyme digestion to obtain a band with a size of 5 300 bp (see FIG. 7, the first figure of the enzyme digestion identification results), which has an expected size, indicating successful enzyme digestion of the recovered fragment. In the figure, 1 and 2 denote pcDNA3.1 enzyme digestion bands. M denotes DL-10 000 Marker.

The digested vectors and fragments are recovered and verified by gel electrophoresis. The plasmid fragment extraction kit is used to extract the target fragments.

The second figure of PD-L1 enzyme digestion identification is as shown in FIG. 8.

2.3 SDS-PAGE Detection

PD-L1 SDS-PAGE detection results are as shown in FIG. 9.

Analysis of the cell culture medium by 10% SDS-PAGE gel electrophoresis shows a size as expected, proving that the protein is successfully expressed, and that the protein is expressed in a soluble form.

2.4 Western-Blot Detection

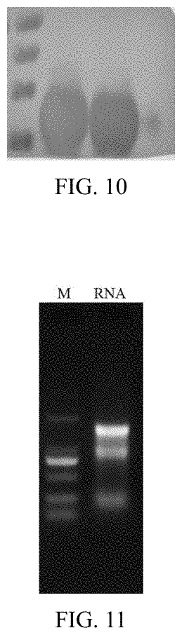

Specific Western-blot detection is as shown in FIG. 10.

Results of Alpaca Immunization Test

| | 12 800 | 25 600 | 51 200 | 102 400 | 204 800 | 409 600 | |
|---|---|---|---|---|---|---|---|
| | folds | folds | folds | folds | folds | folds | PBS |
| PD-L1 before | 0.072 | 0.054 | 0.046 | 0.042 | / | / | 0.041 |
| immunization | | | | | | | |
| PD-L1 of seven | OUT | OUT | 2.242 | 1.491 | 0.784 | 0.395 | 0.043 |
| immunizations | | | | | | | |

This disclosure is further described in combination with the construction of phage library in Experiment 1.

Total RNA Extraction and cDNA Synthesis of Samples:

The results are shown in the gel electrophoresis diagram of total RNA samples in FIG. 11, wherein M is DL 2 000 DNA marker. The four total RNA samples show a very slight degradation. The 28S, 18S, and 5S rRNA bands are clearly visible, and the 28S band brightness is greater than 18S, indicating good RNA integrity. The concentration of RNA samples is measured by Nanodrop, and the results show that the concentration and purity of the RNA samples meet the requirements (Table 1). The cDNA is synthesized by reverse transcription using 10 μg total RNA as a template.

TABLE 1
Concentration and purity of total RNA sample

| | Concentration | |
|---|---|---|
| Sample | (ng/μL) | OD₂₆₀/OD₂₈₀ |
| PD-L1 | 740.9 | 2.04 |

Preparation of VHH Library Fragments:

FIG. 12 shows the electrophoretic analysis of PCR amplification products of camel antibody fragments. In the figure, M is Marker, 1 is a positive control, and 2 is PD-L1. The results are shown in FIG. 12-A: The four samples all have two bands, wherein the main band has a molecular weight of about 600 bp, and there is a non-target band at 900 bp (this band should be an amplified fragment of a traditional antibody). A sufficient amount of PCR products is subjected to electrophoresis, and the main band of 600 bp is recovered by gel extraction, and used as a template for subsequent PCR to amplify VHH using VHH-back and PMCF primers. The results are shown in FIG. 12-B: PCR amplification leads to the obtaining of a target band with a molecular weight as expected (about 400 bp).

In FIG. 12-A, A denotes camel antibody fragment Pt PCR amplification. In FIG. 12-B, B denotes camel nanobody VHH fragment 2^{nd} PCR amplification.

This disclosure is further described in combination with the ligation and transformation pretests in Experiment 1.

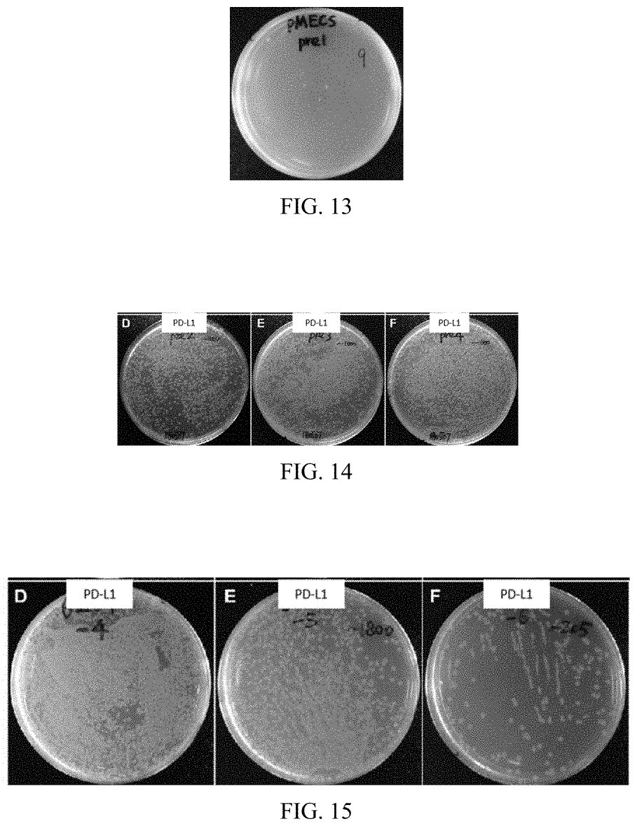

1) The results are shown in FIG. 13 and FIG. 14: Pretest 1 has no inserted fragments, i.e., the vectors are self-ligated, and there are 9 colonies. In the first experimental group of ligation ratio (Pretest 2), the number of clones of PD-L1 is about 1 100. In the second experimental group of ligation ratio (Pretest 3), the number of clones of the four items is about 1600, respectively. In the third experimental group (Pretest 4), the number of clones of the four items is about 1600, respectively. By comparing the number of clones in the vector self-ligation group and that in the optimal experimental group, the ratio of the vector self-ligation can be calculated as: 9÷2 000×100%=0.45%. In the experimental group of the third ligation ratio, the number of clones of the four samples meets the requirements for library construction, and the amount of library fragments is relatively small. Therefore, the ligation ratio of the third experimental group is used for the ligation reaction when the library is formally constructed.

In FIG. 13, after transformation, a culture medium is added to 1 ml for rescue, and 100 μl is coated on an ampicillin-resistant plate.

The vectors and VHH gene fragments are ligated and transformed into E. coli TG1 in different ratios, and the colony growth on the ampicillin-resistant plate is observed. After transformation, a culture medium is added to 1 ml for rescue, and 100 μl is coated on a plate for cultivation and counting, so the number of clones on the plate is one tenth of the actual number of clones.

2) Electrotransformation and Library Construction:

As shown in FIG. 15, the number of clones in the PD-L1 library-5 and -6 gradients are ˜1800 and 205, respectively, so the library capacity is: [205×15×10⁶+(1800+205)×15×10⁵]÷2=3.0×10⁹.

3) Quality Analysis of Immune Library:

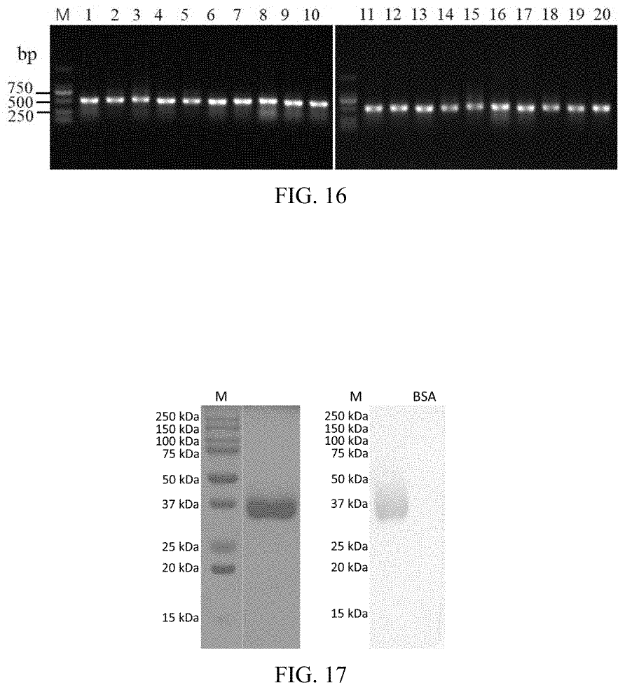

The results are shown in FIG. 16. All clones have specific bands with a molecular weight of about 500 bp, indicating that these clones are positive. These clones are sequenced using primer MP57 (TTATGCTTCCGGCTCGTATG—SEQ ID NO:8).

This disclosure is further described below in combination with materials and methods in the experiment.

1) NeutrAvidin pre-coated boards, Dynabeads and reagents are mainly purchased from companies such as Thermo Scientific and Sinopharm.

2) Helper phage, E. coli TG1 and other experimental materials are preserved by our company.

3) Experimental Methods

PD-L1 Serum Titer Test:

Gradient dilution ELISA method is used to detect the serum titer of PD-L1 before and after immunization; PD-L1 (200 ng/well) is coated; gradient diluted antiserum is added; the secondary antibody is anti-alpaca-HRP used in 1:15 000 dilution; and finally color is developed with a TMB substrate.

PD-L1 Protein Sample SDS-PAGE, Western-Blot and Biotin Labeling:

The loading volume of PD-L1 protein for SDS-PAGE detection is 2 which is subjected to Western-blot detection, antiserum 1:20 000 dilution, and the anti-alpaca-HRP secondary antibody has a working concentration of 1:2 000, and developed by chemiluminescence. The method is the same as described above.

Biotin Labeling and Efficiency Detection of Target PD-L1:

PD-L1 is labeled with biotin under the conditions of 0.25 mg/ml, pH 7.4, protein to biotin ratio of 1:15, and labeled at room temperature for 1 h. The labeled protein is subjected to a PD-Midi desalting column to remove free biotin, and the buffer is replaced with PBS 5% glycerol pH 7.4, and finally packed in aliquots and stored at −70° C. In order to detect the biotin labeling efficiency of PD-L1, two samples of 1.5 μg labeled b-PD-L1 protein are used, and then 5 μg streptavidin (SA) and 5 μl PBS are added, respectively. In addition, 5 μg of SA is also used, to which 5 μl of PBS is added, as a SA sample control. After reacting at room temperature for 1 h, 5 μl of non-reducing loading buffer is added to each sample, and directly subjected to SDS-PAGE without denaturation by heating.

In Vitro Targeted Screening:

PD-L1 is screened for 3 rounds using the constructed immune library.

Identification:

Monoclonal Phage ELISA Analysis

322 clones picked from the enriched products in the second and third rounds of elution are verified by Monoclonal phage ELISA and coated with PD-L1 and BSA control 200 ng/well, respectively.

Soluble ELISA Analysis

Sixteen unique clones of PD-L1 sequence are expressed by IPTG induction at 30° C. After centrifugation, the bacterial cells are collected and subjected to periplasmic cavity extraction. The periplasmic cavity extract sample is diluted 10-fold with 0.5×blocker and added to the coated and blocked PD-L1 and BSA; at the same time, the TG1 (without phagemid) periplasmic cavity extract is set as a negative control. Mouse anti-HA tag monoclonal antibody (ProteinTech, 1:5 000 dilution) is used as a secondary antibody, and goat anti-mouse-HRP (1:5 000 dilution) is used as a tertiary antibody to detect the activity of a soluble expressed nanobody.

4) Construction of pET28a-SUMO Vector Expression and Purification:

In order to increase the expression and activity of nanobodies, 10 clones of active nanobody PD-L1 clones are constructed into pET28a-SUMO vectors for intracellular expression and purified by Ni column after ultrasonic disruption.

The purified nanobodies are diluted with 0.5×blocker gradient and added to the coated and blocked PD-L1 and BSA (200 ng/well), and PBS is set as a negative control. Mouse anti-HA tag monoclonal antibody (ProteinTech, 1:5 000 dilution) is used as the secondary antibody, and goat anti-mouse-HRP (1:5 000 dilution) is used as a tertiary antibody to detect the activity of a soluble purified nanobody.

5) Nanobody Screening:

Antiserum titer determination of target protein PD-L1, SDS-PAGE, Western-blot, biotin labeling and efficiency test. Corresponding immune library is screened for PD-L1, and NeutrAvidin pre-coated ELISA plates (NA-strip) and Invitrogen Dynabeads fixatively biotinylated target PD-L1 (abbreviated as “b-PD-L1”) are used for screening. 3 rounds of screening are performed, and monoclonals are selected for monoclonal phage ELISA tests to identify positive clones for sequencing. The positive clones are subjected to soluble expression, purification and activity analysis.

The antiserum titer of the PD-L1 items is detected by gradient dilution ELISA, and the resulting titer is high, reaching 1:102 400. Western-blot results show that the antiserum can specifically recognize antigen proteins. Protein SDS-PAGE tests show that the protein strips have high purity and no degradation. PD-L1 is subjected to biotin labeling with labeling efficiency >80%.

After three rounds of screening for PD-L1, a significant enrichment appears after the second and third rounds of screening. Monoclonal phage ELISA is used to detect random clones after R3 screening, and the positive rate is about 40%. After sequencing 60 strong positive clones, a total of 16 unique sequence clones are obtained. Analysis of soluble expression activity reveals that 10 clones have good activity. After the clones are constructed into pET28a-SUMO vectors, proteins of five clones are well expressed, and purified nanobodies have good binding activity to PD-L1.

This disclosure is further described below in combination with the table of experimental results of Experiment 1.

PD-L1 Antiserum Titer Test:

Result shows that the antiserum titer is high, reaching 1:102 400.

TABLE 1
Serum titer detection before and after alpaca immunization

| Antiserum | Serum | Serum |
|---|---|---|
| dilution | after | before |
| factor | immunization | immunization |
| 200 | / | 0.631 |
| 400 | OUT | 0.557 |
| 800 | OUT | 0.396 |
| 1600 | OUT | 0.292 |
| 3200 | OUT | 0.183 |
| 6400 | OUT | 0.118 |
| 12800 | OUT | 0.072 |
| 25600 | OUT | 0.054 |
| 51200 | 2.242 | 0.046 |
| 102400 | 1.491 | 0.042 |
| 204800 | 0.784 | / |
| 409600 | 0.395 | / |
| PBS | 0.043 | 0.041 |

OUT denotes that the ELISA value at OD450 nm is greater than 3.

PD-L1 protein sample SDS-PAGE, Western-blot and biotin labeling:

1) Screening Antigen SDS-PAGE and Western-Blot Detection:

The target protein PD-L1 is detected by SDS-PAGE. The results are shown in FIG. 17, indicating high protein purity, no degradation, and a molecular weight of about 35 kDa, which meets the requirements of subsequent labeling and screening. Western-blot test results show that antiserum specifically recognizes PD-L1.

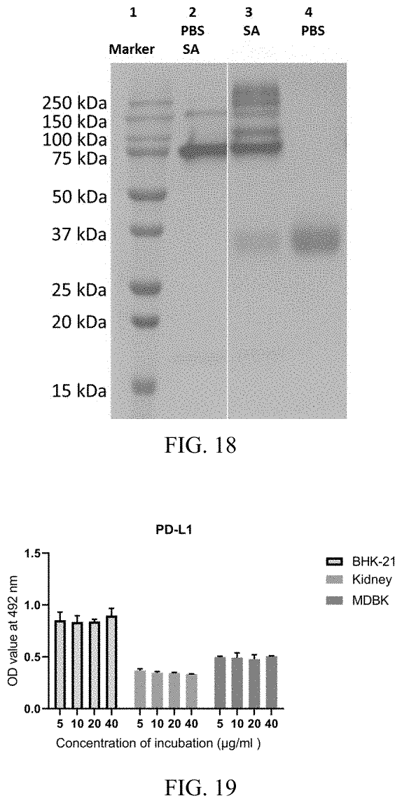

2) The results of biotin labeling efficiency detection are shown in FIG. 18, indicating that the SA+b-PD-L1 lane has a significant band migration than SA+PBS, while there is a significant reduction in the b-PD-L1 band near 35 kDa than an equal amount of b-PD-L1+PBS group, so the labeling efficiency is estimated to be >80%.

3) In Vitro Targeted Screening

The b-PD-L1 is screened for 3 rounds using the constructed immune library. The results are as shown in the following table:

| | | | | Enrich- |
|---|---|---|---|---|
| | | | | ing |
| Round | Conditions | Input | Output | factor |
| 1^{st}-P | Target protein: b-PD-L1 (10 μg) | 1.0 × 10¹³ | 1.1 × 10⁸ | 9.1 × |
| | Blocking: 2% Milk-PBS | | | 10⁴ |
| | Washing: 0.1% Tween 20-PBS, | | | |
| | 10 times | | | |
| | Elution: 0.2 M Glycine-HCl, | | | |
| | pH 2.2 | | | |
| | Pre-counter select: None | | | |
| 2^{nd}-P | Target protein: b-PD-L1 (5 μg) | 5.6 × 10¹¹ | 6.0 × 10⁶ | 9.3 × |
| | Blocking: 2% Milk-PBS | | | 10⁴ |
| | Washing: 0.2% Tween 20-PBS, | | | |
| | 15 times | | | |
| | Elution: 0.2 M Glycine-HCl, | | | |
| | pH 2.2 | | | |
| | Pre-counter select: None | | | |
| 3^{rd}-P | Target protein: b-PD-L1 (1 μg) | 1.4 × 10¹² | 2.0 × 10⁸ | 7.0 × |
| | Blocking: 2% Milk-PBS | | | 10³ |
| | Washing: 0.2% Tween 20-PBS, | | | |
| | 20 times | | | |
| | Elution: 0.2 M Glycine-HCl, | | | |
| | pH 2.2 | | | |
| | Pre-counter select: None | | | |

The screening results show that there is a significant enrichment in the second and third rounds of screening (R2 and R3) (the high output of the first round is caused by the non-specific adsorption of NA strip).

4) Identification

Phage ELISA Detection.

| Number | PD-L1 (200 ng/well) | BSA (200 ng/well) |
|---|---|---|
| PD-L1-7 | 0.635 | 0.054 |
| E. coli TG1 | 0.046 | 0.049 |

The following screening tests will be conducted in conjunction with specific detection tests:

1 Method

1.1 Nanobody Cytotoxicity Experiment

BHK-21 cells, MDBK cells, and goat kidney cells are rescued in 37° C. warm water; cell culture medium (90% DMEM+10% FBS) is added, and passaged to 96-well plates, 6×10³ cells per well. The cells are left to adhere and incubate for 3 hours. PD-L1 nanobodies are added, and the final concentration gradient is set to be: 5 μg/ml, 10 μg/ml, 20 μg/ml, 40 μg/ml. There are 4 gradients in total. 20 μl/well of MTS reagent is added to each well and incubated at 37° C. for 3 hours. After shaking, absorbance is measured at 492 nm.

1.2 Nanobody and Phage NO Detection Experiment

Mouse immune cells are added to the 96-well plate; 3 h later, T7 phage with a final concentration of 1 μg/ml is added, and then incubated for 24 h. The PD-L1 nanobodies are added to the wells after diluted with the culture medium (90% DMEM+10% FBS). The final concentration is adjusted to 10 μg/ml, 20 μg/ml, 40 μg/ml, 80 μg/ml, three replicate wells per sample. Cells are cultured in a cell incubator for 38 h. 100 μl extraction solution, 50 μl reagent 1, and 50 μl reagent 2 (both are reagents in the NO level detection kit purchased from Beijing Solarbio Co., Ltd.) are added to the control group. 100 μl sample, 50 μl reagent 1 and 50 μl reagent 2 are added to the experimental group to be detected, evenly mixed, and then left to stand still at room temperature for 15 min to measure the absorbance at 550 nm.

1.3 Data Analysis

The results are expressed as mean±standard deviation (SD). Statistical analysis is performed using GraphPad Prism 8 software, and significant difference analysis is performed by Mann-Whitney U test (*=P<0.05, *=P<0.01).

1.4 Changed Levels of IL-4, IFN-γ Cytokines and NO Secretion in Mice After Injection of Nanobodies

Antibody-injected BalB/C mice, 0.1 mg each. Each is injected with 0.2 ml with a concentration of 0.5 mg/ml. The grouping is as follows: (For example, there are two groups in the PBS control group, which are not duplicates and errors. The subsequent challenge is performed with Staphylococcus aureus and Streptococcus agalactiae, respectively, so there are two groups for each antibody injection).

Antibody injection in groups

| | |
|---|---|
| PBS control | 8 mice |
| PD-L1 | 8 mice |
| PBS control | 8 mice |
| PD-L1 | 8 mice |

Three days after immunization, blood is collected and serum is separated to detect the levels of cytokines IL-4, IFN-γ and NO level (IL-4 detection kit, IFN-γ detection kit and NO level detection kit are all purchased from Beijing Solarbio Co., Ltd.).

1.5 Protection Experiments Against Staphylococcus aureus and Streptococcus agalactiae Challenge

Then Staphylococcus aureus and Streptococcus agalactiae are separately injected, wherein Staphylococcus aureus is injected in a dose of 150 μl/1.9×10⁹ cfu (the lowest lethal dose of mice determined by multiple studies). Streptococcus agalactiae is injected in a dose of 200 μl/5.1×10¹⁰ cfu (the lowest lethal dose of mice determined by multiple studies). After 24 hours, the state of mice is observed and recorded.

2 Results

2.1 Cytotoxicity Determination of Nanobodies

As shown in FIG. 19, the cytotoxicity of PD-L1 nanobody BHK-21 cells, sheep kidney cells, and MDBK cells is shown. In the figure, the abscissa is the treatment of different concentrations of PD-L1 (μg/ml), and the ordinate is the OD value at 492 nm. BHK-21 is the experimental group of BHK-21 cells. Kidney is the experimental group of sheep kidney cells, MDBK is the experimental group of MDBK cells, and the data is expressed as mean ±SD.

Nanobodies of different concentrations are not cytotoxic to mice (BHK-21 cells), sheep (sheep kidney cells) and bovine cells (MDBK cells). It is proved that PD-L1 nanobodies are not cytotoxic to animal cells.

MTT with trade name of thiazole blue can be used to quantitatively measure and analyze cell survival and growth by spectrophotometry at a specific wavelength. MTS cell proliferation analysis kit of BioVision is a colorimetric method and is an upgraded version of MTT. It is used to sensitively quantify live cells in proliferation and cytotoxicity analysis, and can be used to determine whether the reagent is toxic to cells. By verification, it shows that nanobodies are not toxic to mammalian cells BHK-21, MDBK or sheep kidney cells, and can be safely used in animals.

2.2 NO Activation Test

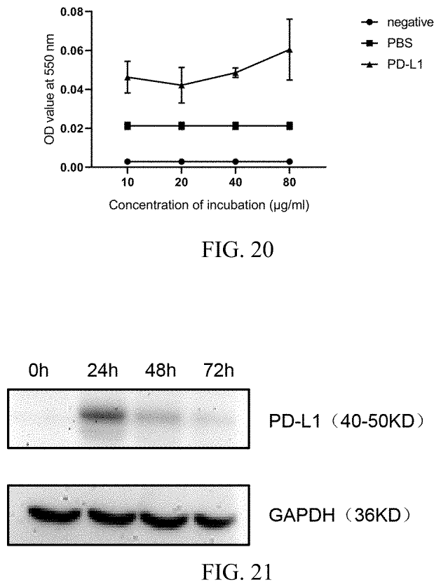

As shown in FIG. 20, the NO enhancement effect of PD-L1 on primary cells after the induction of mouse bone marrow stem cells is shown. In this figure, negative is a negative control of the kit; PBS is a negative control with PBS addition, PD-L1 is the experimental group with addition of PD-L1 nanobodies.

After the T7 phage is added to the primary cells after the induction of mouse bone marrow stem cells, PD-L1 nanobodies of different concentrations are added and incubated for 24 h; it is shown that the enhancement of PD-L1 nanobodies on NO concentration is positively correlated with the concentration; the higher the PD-L1 antibody concentration, the larger the NO detection value. The maximum value is reached at 80 μg/ml.

Nitric oxide (NO), as an intercellular and intracellular information-transmitting substance, plays a role in signal transmission and is a new type of biological messenger molecule. Studies of the State Key Laboratory of Veterinary Pathogen Biology and the Key Laboratory of Animal Virology of the Ministry of Agriculture, Lanzhou Veterinary Research Institute, Chinese Academy of Agricultural Sciences show that T7 phage particles are easily engulfed by immune cells and promote the maturation of immune cells and the secretion of NO and other cytokines, which can be used as a cell model to verify the interaction relationship between FMDV and immune cells and intensity thereof. The NO content in the culture medium is detected with a kit to reflect the intensity of interaction between immune cells and antigens.

PD-L1 nanobodies at a high concentration have a better ability to promote the level of NO secreted by immune cells, and release the suppression of other immune cells to immune cells such as T cells (by the binding of PD-L1 receptors and ligands). The ability to promote immune cells shows that PD-L1 nanobodies can be used to promote the improvement of animal cell immune levels, and can be used as a potential immune adjuvant or immune promoter when there is virus transmission. PD-L1 immunosuppressive receptors also exist in other immune-related cells, and it has been verified that blocking immune checkpoints can enhance the activity of immune-related cells.

2.3 Changed Levels of IL-4, IFN-γ Cytokines and NO Secretion in Mice After Injection of Nanobodies

Solarbio IL-4 cytokine detection kits are used to detect IL-4 cytokine levels in mouse serum; Solarbio IFN-γ cytokine detection kits are used to detect IFN-γ cytokine levels in mouse serum; Solarbio NO detection kits are used to detect NO levels in mouse serum. The results are shown in the table below. (8 mice are tested in each group and each data represents the OD450 value detected in 1 mouse.)

| IL-4 | | IFN-γ | | NO | |
|---|---|---|---|---|---|
| PBS | PD-L1 | PBS | PD-L1 | PBS | PD-L1 |
| 0.093 | 0.119 | 0.071 | 0.063 | 0.066 | 0.072 |
| 0.088 | 0.128 | 0.069 | 0.062 | 0.075 | 0.074 |
| 0.08 | 0.122 | 0.076 | 0.062 | 0.072 | 0.066 |
| 0.096 | 0.099 | 0.091 | 0.069 | 0.08 | 0.076 |
| 0.093 | 0.083 | 0.077 | 0.057 | 0.073 | 0.061 |
| 0.105 | 0.078 | 0.083 | 0.064 | 0.077 | 0.064 |
| 0.087 | 0.094 | 0.088 | 0.068 | 0.073 | 0.063 |
| 0.124 | 0.099 | 0.12 | 0.065 | 0.082 | 0.066 |

2.4 Protection Experiments Against Staphylococcus aureus and Streptococcus agalactiae Challenge

Each group has 8 mice, and the state of mice after 24 hours of challenge is shown in the table below.

| | | Good | Bad | Death/ |
|---|---|---|---|---|
| | Mice/8 in | state/number | state/number | number |
| | each group | of mice | of mice | of mice |
| Challenge | PBS | | 1 | 7 |
| with S. | PD-L1 | | 5 | 3 |
| aureus | | | | |
| Challenge | PBS | | 1 | 7 |
| with S. | PD-L1 | | 2 | 6 |
| agalactiae | | | | |

The ability to promote the immune level of mice reveals that PD-L1 nanobodies can be used to promote the improvement of immune levels of animal cells, and can be used as a potential immune adjuvant or an immune promoter for animal protection when viruses/bacteria are transmitted. PD-L1 immunosuppressive receptors also exist in other immune-related cells. Although many mechanisms have not been studied, it has been clearly verified that blocking immune checkpoints can enhance the activity of immune-related cells. The PD-L1 nanobodies prepared in this study can be used to protect animals against infection by Staphylococcus aureus and Streptococcus agalactiae and increase the number of mice that survive.

2.5 Results and Method of Flow Cytometric Detection of PD-L1 Nanobodies:

PD-L1 receptors are expressed using MC-38 cells, and the PD-L1 expression is shown in FIG. 21.

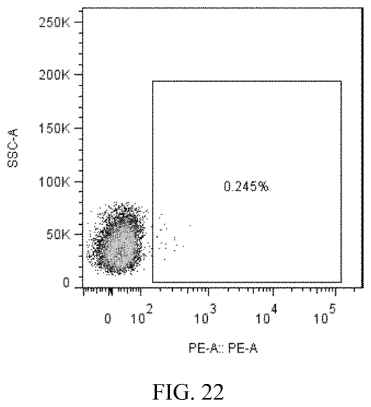

The flow detection steps are usual steps. The flow cytometric results of PD-L1 nanobodies and MC-38 cells are shown in FIG. 22.

The above are only preferred examples of this disclosure and are not intended to limit this disclosure. Any modification, equivalent replacement and improvement made within the spirit and principle of this disclosure should be included in the protection scope of this disclosure.

1.-10. (canceled)

11. A nanobody, wherein the nanobody is a PD-L1 nanobody having a sequence of SEQ ID NO: 1, and a DNA sequence of:
CGGGGCGGGAACATTTCCAAGCTTAAGGAGACAGTACATATGAAATACCT
ATTGCCTACGGCAGCCGCTGGATTGTTATT
ACTCGCGGCCCAGCCGGCCATGGCCCAGGTGCAGCTGCAGGAGTCTGGGG
GAGGCTTGGTGCAGGCTGGGGGCTCTCTGA
GACTCTCCTGTGCAGCCTCTGGACGCACCTTCAGAAACGATGTCATGGCC
TGGTTCCGCCAGATTCCAGGGAAGGAGCGT
GAGTTTGTTGCGGTGATTGCCTACGATGCGGCTGACACAGACTACGCAGA
CTCCGTGAAGGGCCGATTCATCATCTCCAG
AGACAACGCCAAGAACACGATATATTTGCAAATGAACACCCTGAAACCTG
AGGACACGGCCGTTTATTACTGTGCAGCCG
ACAAGGACAGAATGTACGGTAGTAGGCACTGGCCGGAATATGAGTATGAC
TACTGGGGCCAGGGGACCCAGGTCACCGTC
TCCTCAGCGGCCGCATACCCGTACGACGTTCCGGACTACGGTTCCCACCA
CCATCACCATCACTAGACTGTTGAAAGTTG
TTTAGCAAAACCTCATACAGAAAATTCATTTACTAACGTCTGGAAAGACG
ACAAAACTTTAGATCGTTACGCTAACTATG
AGGGCTGTCTGTGGAATGCTACAGGCGTTGTCGTTTGTACTGGTGACGAA
ACTCAGTGTTACGGTACATGGGTTCCTATT
GGGCTTGCTATCCCTGAAAATGAGGGTGGTGGCTCTGAGGGTGGCGGTTC
TGAGGGTGGCGGTTCTGAGGGTGGCGGTAC
TAAACCTCCTGAGTACGGTGATACACCTATTCCGGGCTATACTTATATCA
ACCCTCTCGACAGCACTTATCCGCCTGGTA
CTGGAGCAAAACCCCGCTAATCCTAAATCCTTCTCTTGGAGGAGTCTCAG
CCTCTTAATACTTTCATGTTTCAGAATAAT
AGGTCCGAATAAGGCAGGGTGCATAAGCTGTTTATACGGGACTGTTACTC
AAGGCACTGACCCGATTAAAGTTAGTAACA
GTACACTCCCGTGAATCATACGAAGCATGGTAGGACGCTTAACTGGGACA
GGAAAAGTC.

12. The method for preparing PD-L1 nanobodies from an alpaca immunized with PD-L1 antigens expressed in mammalian cells according to claim 11, wherein the method for preparing PD-L1 nanobodies from an alpaca immunized with PD-L1 antigens expressed in mammalian cells further comprises:
step 1: construction of a vector: amplification of a target fragment, replacement enzyme digestion, ligation of target fragment and vector, transformation, and screening of clones;
step 2: protein identification and expression;
step 3: construction of an antibody library: total RNA extraction and cDNA synthesis of samples, preparation of VHH library fragments, electrotransformation and library construction;
step 4: biotinylation screening and prokaryotic expression.

13. The method for preparing PD-L1 nanobodies from an alpaca immunized with PD-L1 antigens expressed in mammalian cells according to claim 12, wherein in the step 1, the amplification of the target fragment comprises:
(1) designing synthetic primers:
PD-L1 upstream primer;
5′-GACACGAATTCGCCACC-3′
PD-L1 downstream primer;
5′-GTGTCAAGCTTTCACTTATCATCA-3′
amplifying a sufficient amount of target product by PCR;
(2) using a pfu high-temperature polymerase for PCR reaction;
in the step 1, the specific steps of PCR amplification of target fragment include:
(1) a first round PCR procedure:

| | | |
|---|---|---|
| 95° C. 3 min | | |
| 95° C. 22 sec | | |
| | {close oversize brace} | 18 cycles; |
| 50° C. 20 sec | | |
| 72° C. 40 sec | | |
| 72° C. 5 min | | |

the first round PCR product is used as the template for a second round PCR;
(2) a second round PCR reaction system:
the amount of components of PCR is as follows: The primer concentration is 1 OD soluble in 400 μl of ddH₂O;

| | |
|---|---|
| Upstream primer-1 | 2 μl |
| Downstream primer-34 | 2 μl |
| First round PCR product | 1 μl |
| dNTP | 1 μl (25 mM each) |
| 10 × pfu Buffer | 5 μl |
| Pfu | 0.4 μl (5 U/μl) |
| ddH₂O | To 50 μl |

(3) a second round PCR procedure:

| | | |
|---|---|---|
| 95° C. 3 min | | |
| 95° C. 22 sec | | |
| | {close oversize brace} | 22 cycles; |
| 55° C. 20 sec | | |
| 72° C. 45 sec | | |
| 72° C. 5 min | | |

the second round of PCR is subjected to agarose gel electrophoresis to recover purified fragments for enzyme digestion.

14. The method for preparing PD-L1 nanobodies from an alpaca immunized with PD-L1 antigens expressed in mammalian cells according to claim 12, wherein in the step 1, the recovered purified target DNA fragments are ligated to vectors,
ligation system: 20 μl;
enzyme-digested target fragment: 8 μl;
enzyme-digested vector PCDNA3.1+4 μl;
10×T4 DNAligase Buffer 2 μl;
T4 DNAligase 1 μl (5 U/μl);
ddH₂O supplemented to 20 μl;
the above ligation mix solution is placed in a PCR machine at 22° C. for 1 h.

15. The method for preparing PD-L1 nanobodies from an alpaca immunized with PD-L1 antigens expressed in mammalian cells according to claim 12, wherein in the step 1, transformation and screening of clones are carried out:
the above ligation solution is transferred into oneshort competent cells to detect and screen positive clones for sequencing.

16. The method for preparing PD-L1 nanobodies from an alpaca immunized with PD-L1 antigens expressed in mammalian cells according to claim 12, wherein step 4 comprises:
1) PD-L1 Serum Titer Test:
the serum titer of PD-L1 before and after immunization is detected by gradient dilution ELISA method comprising coating with PD-L1, adding with gradient diluted antiserum, using a secondary antibody which is anti-alpaca-HRP 1:15 000 dilution, and finally developing with a TMB substrate;
2) PD-L1 Protein Sample SDS-PAGE, Western-Blot and Biotin Labeling:
the loading volume of PD-L1 protein for SDS-PAGE detection is 2 which is subjected to Western-blot detection, antiserum 1:20 000 dilution, and the anti-alpaca-HRP secondary antibody has a working concentration of 1:2 000, and developed by chemiluminescence;
3) Biotin Labeling and Efficiency Detection of Target PD-L1:
PD-L1 is labeled with biotin under the conditions of 0.25 mg/ml, pH 7.4, protein to biotin ratio of 1:15, and labeled at room temperature for 1 h; the labeled protein is subjected to a PD-Midi desalting column to remove free biotin, and the buffer is replaced with PBS 5% glycerol pH 7.4, and finally packed in aliquots and stored at −70° C.;
detection of biotin labeling efficiency of PD-L1: Two samples of 1.5 μg labeled b-PD-L1 protein are used, and then 5 μg streptavidin and 5 μl PBS are added, respectively. In addition, 5 μg of SA is also used, to which 5 μl of PBS is added, as a SA sample control; after reacting at room temperature for 1 h, 5 μl of non-reducing loading buffer is added to each sample, and directly subjected to SDS-PAGE without denaturation by heating;
4) In Vitro Targeted Screening:
PD-L1 is screened for 3 rounds using the constructed immune library;
5) Identification:
322 clones picked from the enriched products in the second and third rounds of elution are verified by Monoclonal phage ELISA and coated with PD-L1 and BSA control 200 ng/well, respectively;
16 unique clones of PD-L1 sequence are expressed by IPTG induction at 30° C.; after centrifugation, the bacterial cells are collected and subjected to periplasmic cavity extraction; the periplasmic cavity extract sample is diluted 10-fold with 0.5×blocker and added to the coated and blocked PD-L1 and BSA; at the same time, the TG1 periplasmic cavity extract is set as a negative control; mouse anti-HA tag 1:5 000 diluted monoclonal antibody is used as a secondary antibody, and 1:5 000 diluted goat anti-mouse-HRP is used as a tertiary antibody to detect the activity of a soluble expressed nanobody.

17. The method for preparing PD-L1 nanobodies from an alpaca immunized with PD-L1 antigens expressed in mammalian cells according to claim 12, wherein step 3 comprises:
four VHH library fragments are amplified using camel antibody primer pairs, respectively;
the four VHH library fragments are inserted into pMECS phagemid vectors respectively and introduced into E. coli TG1 to construct a phage display antibody immune library with a storage capacity of >10⁹; 20 clones are randomly picked from the library for sequence determination and analysis, so that more than 99% of the clones in the library comprise the target insert sequence.

18. Use of the nanobody according to claim 11 in the preparation of a reagent for tumor detection or treatment, an immune adjuvant for improving the immune level of an animal or of an immune promoting agent during the transmission of viruses and/or bacteria.
